(19) United States
(12) Patent Application Publication
(10) Pub. No.: US 2025/0051774 A1
(43) Pub. Date: Feb. 13, 2025

(54) DIRECT PRODUCTION OF SIRNAS IN SACCHAROMYCES BOULARDII AND PACKAGING IN EXTRACELLULAR VESICLES (EVs) FOR TARGETED GENE SILENCING

(71) Applicant: Mercury Bio, Inc., Santa Fe, NM (US)

(72) Inventors: Tatiana Vinogradova-Shah, Rochester, NY (US); Richard T. Sayre, Los Alamos, NM (US); Alex Pertzev, Santa Fe, NM (US); Nianzi Liu, Boulder, CO (US); Arle Kruckeberg, Santa Fe, NM (US)

(21) Appl. No.: 18/895,210

(22) Filed: Sep. 24, 2024

Related U.S. Application Data
(63) Continuation-in-part of application No. PCT/US23/64756, filed on Mar. 21, 2023.
(60) Provisional application No. 63/323,198, filed on Mar. 24, 2022.

(51) Int. Cl. C12N 15/113; A61K 9/50; C12N 15/81

(52) U.S. Cl. CPC C12N 15/113; A61K 9/5068; C12N 15/81; C12N 2310/14

(57) ABSTRACT

The present invention is direct to the direct expression of siRNA molecules in yeast, which can be packaged into extracellular vesicles (EVs) and delivered to a target organism causing the downregulation of a select gene.

### CROSS-REFERENCE TO RELATED APPLICATIONS

This International PCT Application claims the benefit of and priority to U.S. Provisional Application No. 63/323,198, filed Mar. 24, 2022. The entire specification, claims, and figures of the above-referenced application is hereby incorporated, in its entirety by reference.

### SEQUENCE LISTING

The instant application contains contents of the electronic sequence listing Corrected-90355-00062-Sequence-Listing.xml; Size: 26,027 bytes; and Date of Creation: Nov. 1, 2024) is herein incorporated by reference in its entirety.

### TECHNICAL FIELD

The present invention is directed to the field of molecular biology, and in particular the production and processing of heterologous RNA molecules in yeast cells. Specifically, the invention is directed to the heterologous expression of dsRNA molecules in yeast host cells that are processed into siRNA by a membrane dicer enzyme, and their subsequent packaging into extracellular vesicles (EVs) for delivery to a target cell.

### BACKGROUND

Several yeast species, e.g., Saccharomyces cerevisiae and S. boulardii, lack the molecular machinery necessary to produce small interfering RNA species (siRNA) and to silence genes using this machinery (Drinnenberg et al., 2009). This inability to utilize RNA interference to regulate gene expression is primarily due to the lack of the genes encoding the dicer and argonaut proteins of the RNA induced silencing complex (RISC). Recently, it has been demonstrated that the expression of foreign genes involved in RNA-induced silencing can restore functional RNA interference activity and targeted gene silencing in recombinant S. cerevisiae (reviewed in Chen et al., 2020). Reconstitution of a functional RNA interference system in S. cerevisiae required only three genes: Argonaute-2 (Ago2), Dicer, and the HIV-1 transactivating response RNA-binding protein (Suk et al., 2011). Ago2 binds the guide siRNA and cleaves the mRNA target. The dicer gene processes dsRNA precursors into siRNA, and the HIV-1 transactivating response RNA-binding protein facilitates RNA binding to the protein complex.

More recently, a novel class of dicer proteins was identified in the yeast, S. castellii. Unlike other known dicer enzymes, for example those in Schizosaccharomyces pombe, plants and animals, the S. castellii dicer protein has only a single RNase III RNA cleavage domain, rather than two RNase III domains, and no helicase or Piwi Argonaute Zwille (PAZ) domains. It has been proposed that the S. castellii dicer may act as a homodimer with two RNA cleavage domains. In addition, the S. castellii dicer protein appears not to require additional dsRNA binding proteins, thus representing a simpler RNA induced silencing complex than that found in plants and animals (Drinnenberg et al., 2009). Unlike canonical dicers, which generate siRNAs of regular sizes beginning from the termini of dsRNAs, the S. castellii dicer begins processing dsRNA in the interior of the molecule and works outward with a product size determined by the distance between the two neighboring active sites of the dicer enzyme complex (Weinberg et al., 2011; Wilson and Doudna, 2013).

Due to S. casteliii's evolutionary closeness to other yeast species and its simplicity, elements of the S. castellii RNAi pathway are commonly employed for expressing full RNA silencing activity in S. cerevisiae (Drinnenberg et al., 2009; Crook et al., 2014; Si et al., 2014; Williams et al., 2015b; Purcell et al., 2018; Wang et al., 2019). Additional factors that enhance RISC activity in recombinant S. cerevisiae include the use of strong constitutive yeast gene promoters, such as PTEF1, PGPD1, PTPI1, and PPGK1 to drive high level expression of the dicer and AGO genes. This requirement for high level dicer and AGO expression for functional RNA interference activity was also confirmed by Kildegaard et al., (2019), who observed that the use of the strong yeast gene promoter, PTDH3, to drive short hairpin RNA (shRNA) expression elicited higher RNA repression levels relative to the weak PRNR2 promoter when targeting an endogenous yeast gene (ZWF1) for silencing.

RNA hairpin length was also shown to affect RNA silencing efficiency. When the length of shRNA was increased from 100 bp to 200 bp, gene silencing efficiency was improved by 30% when targeting a strongly expressed yellow fluorescent protein (YFP) in S. castellii (Crook et al., 2014). Notably, when YFP was only weakly expressed, the RNAi silencing effect of a 200 bp shRNA was 6-fold higher than that of the 100 bp shRNA, further indicating that longer hairpin length is especially important for efficient silencing of low-abundance transcripts (Williams et al., 2015a; Kildegaard et al., 2019).

As shown below, the present inventors demonstrate the heterologous expression and processing of RNA molecules into siRNA, which is further packaged into extracellular vesicles (EVs) for delivery to a target cell causing the downregulation of a target gene.

### SUMMARY OF THE INVENTION

The present invention include systems, methods, and compositions for the direct expression of siRNA molecules in yeast, which can be packaged into extracellular vesicles (EVs) and delivered to a target organism causing the downregulation of a target gene.

In one aspect, the present invention is directed to EVs derived from Saccharomyces, such as Saccharomyces cerevisiae (sometimes referred to herein as Sc) or Saccharomyces boulardii (sometimes referred to herein as Sb) containing a heterologous siRNA molecule configured to down-regulate a target gene in a host organism.

The present invention is directed to EVs derived from Saccharomyces, such as Saccharomyces cerevisiae or Saccharomyces boulardii co-expressing a heterologous long shRNA and one or more peptides configured to generate siRNAs from the heterologous shRNA.

The present invention is directed to EVs derived from Saccharomyces, such as Saccharomyces cerevisiae or Saccharomyces boulardii expressing a dicer peptide derived from S. castellii S. castellii, or a fragment or variant thereof.

The present invention is directed to EVs derived from Saccharomyces, such as Saccharomyces cerevisiae or Saccharomyces boulardii co-expressing a heterologous long shRNA and a dicer peptide derived from S. castellii, or a fragment or variant thereof, configured to generate siRNAs from the heterologous shRNA.

In one preferred aspect, the invention includes a genetically modified yeast cell co-expressing a heterologous dicer peptide and a shRNAs configured to down-regulate a target gene in a host organism. In this preferred aspect, heterologously expressed RNA of the invention may include the long shRNA that is at least 30 or more base pairs in length, and may further be processed by the co-expressed heterologous dicer into discrete siRNAs for packaging and delivery to a host organism through one or more EVs.

In another preferred aspect, the invention includes a genetically modified yeast cell co-expressing a heterologous dicer peptide derived from S. castellii, or a fragment or variant thereof, and a shRNAs configured to down-regulate a target gene in a host organism. In this preferred aspect, heterologously expressed RNA of the invention may include the long shRNA that is at least 30 or more base pairs in length, and may further be processed by the co-expressed heterologous dicer into discrete siRNAs for packaging and delivery to a host organism through one or more EVs derived from S. cerevisiae and S. boulardii.

In another preferred aspect, the invention includes a genetically modified yeast cell co-expressing a heterologous dicer peptide derived from S. castellii or a fragment or variant thereof that is fused to the C-terminus of a yeast EV membrane protein such as Sur7 (SEQ ID NO. 5), and/or a truncated Fet3 (SEQ ID NO. 9) or a truncated Msb2 (SEQ ID NO. 8) protein, or the Glycophorin A transmembrane spanning domain comprising residues 92-114 (SEQ ID NO. 7), such that the dicer enzyme is localized in the lumen of the EV to process shRNAs localized to EVs into siRNAs.

Additional aspects of the invention include one or more expression vectors having a nucleotide sequence, operably linked to a promoter, encoding a heterologous dicer peptide, or a fragment or variant thereof, and a shRNAs configured to down-regulate a target gene in a host organism.

In another aspect, the present invention includes systems, methods and compositions for the production of Saccharomyces-generated EVs (sometimes referred to as exosome or SGEVs) from cells co-expressing a heterologous dsRNA and a dicer peptide configured to produce therapeutic RNAs, particularly small interfering RNA (siRNA) for the treatment of a disease or condition in a subject in need thereof. In another aspect, the present invention includes systems, methods and compositions for the production of Saccharomyces-generated EVs (sometimes referred to as exosome or SGEVs) configured for the delivery of therapeutic RNA, particularly small interfering RNA (siRNA) for the treatment of a disease or condition in a subject in need thereof.

Additional aspects of the invention include transforming a yeast cell to expression one or more of the expression vectors of the invention. Still further aspects of the invention include one or more isolated EVs of the invention containing siRNA generated by a yeast cell expressing an expression vectors of the invention.

Additional aspects of the invention include systems, methods and composition for the production and processing of RNA molecules in yeast cells, resulting in yeast-derived EVs packed with siRNAs configured to downregulate expression of a target gene, preferably in vivo or in vitro. In this preferred aspect, the invention include systems, compositions, and methods for making a quantity of isolated yeast-generated extracellular vesicle (EVs) containing one or more heterologous small interfering RNAs (siRNAs). As described below, the yeast-cell from which the EVs are derived, which is preferably selected from Saccharomyces cerevisiae, or Saccharomyces boullardi, can also express a heterologous fusion peptide having a first dicer protein domain fused to an second EV membrane protein domain. In this aspect, the double-stranded RNA (dsRNA) expressed in the yeast cell are processed into said siRNAs by a heterologously expressed fusion peptide, which may preferably be embedded in the membrane of an EV, such that the siRNAs are retained in the EV and subsequently isolated for therapeutic or diagnostic uses. In one example, a an siRNA according to the nucleotide sequence SEQ ID NO. 14 can be used to target a gene of SARS-CoV-2.

In another preferred aspect the dsRNA expressed in the yeast-cell can include a long dsRNA, which can have between 30 and 250 more base pairs, or in alternative embodiment a long dsRNA of the invention can include more than 250 more base pairs. In another preferred aspect, the long dsRNA is configured to form a hairpin RNA (hpRNA). An exemplary hpRNA of the invention can be directed to downregulate expression of the nsp2 gene and 5′ region in SARS-CoV-2 and can include the to the nucleotide sequence according to SEQ ID NO. 10, or a fragment thereof.

In a preferred aspect of the invention, a heterologous fusion peptide having a dicer protein domain can include a dicer protein dicer protein from Saccharomyces castellii, and preferably a dicer protein derived from S. castellii according to the amino acid sequence SEQ ID NO. 1, or a fragment or variant thereof, or a dicer or dicer like proteins from Saccharomyces uvarum (SEQ ID NO. 3), or Naumovozyma dairenensis (SEQ ID NO. 4). Additional exemplary EV fusion proteins that can form the second EV membrane domain can include the a yeast EV membrane protein such as Sur7 (SEQ ID NO. 5), and/or a truncated Fet3 (SEQ ID NO. 9) or a truncated Msb2 (SEQ ID NO. 8) protein, or the Glycophorin A transmembrane spanning domain comprising residues 92-114 (SEQ ID NO. 7). (Notably, as used herein, the term “dicer-like” generally can be interchangeably referred to as a Dicer).

Additional exemplary EV fusion proteins of the invention can include a fusion peptide of the invention can include a first dicer protein domain comprising a dicer protein from S. castellii, or a fragment or variant thereof, and said second EV membrane protein domain comprising an Sur7 protein from S. cerevisiae, or a fragment or variant thereof. In another one example, a fusion peptide of the invention can include a dicer protein domain comprising a dicer protein from S. castellii (SEQ ID NO. 1), S. uvarum (SEQ ID NO. 3) or Naumovozyma dairenensi (SEQ ID NO.4), or a fragment or variant thereof, and said second EV membrane protein domain comprising a yeast EV membrane protein such as Sur7 (SEQ ID NO. 5), and/or a truncated Fet3 (SEQ ID NO. 9) or a truncated Msb2 (SEQ ID NO. 8) protein, or the Glycophorin A transmembrane spanning domain comprising residues 92-114 (SEQ ID NO. 7).

Alternative examples of fusion peptides of the invention can further include a fusion peptide including a first dicer protein domain comprising a dicer protein according to the amino acid sequence SEQ ID NO. 1, 3-4, or a fragment or variant thereof, and said second EV membrane protein domain comprising a EV membrane protein according to the amino acid sequence SEQ ID NO. 5, 7-9, or a fragment or variant thereof. In another example, a fusion peptide of the invention can include a dicer peptide according to the amino acid sequence SEQ ID NO. 1, 3-4, or a fragment or variant thereof, and an EV membrane peptide according to the amino acid sequence SEQ ID NO. 5, 7-9, or a fragment or variant thereof;

In certain aspects, the first and second domains of the fusion peptide of the invention can be fused by a linker, and preferably a peptide linker such as a (GGGS)₃ linker according to SEQ ID NO. 12.

Additional aspects of the invention will be evident from the specification, figures, and claims provided herein.

### BRIEF DESCRIPTION OF THE DRAWINGS

FIG. 1A-1B. Cassettes for expression of dicer and dsRNA targeting nsp1 sars-CoV2 gene (FIG. 1A) or dsRNA without a dicer (FIG. 1B).

FIG. 2A-2B. Northern blot analysis of nsp1 dsRNA in yeast cells and EVs. (FIG. 2A) RNA gel before transfer; (FIG. 2B) membrane hybridized with probes for nsp1 gene. Gel lines: 1—ssRNA ladder; 2—miRNA from wt yeast cells; 3—miRNA from Sb-DCR1-dsNsp1 cells; 4—miRNA from Sb-dsNsp1 cells; 5—miRNA from wt EVs; 6—miRNA from Sb-DCR1-dsNsp1 EVs; 7—miRNA from Sb-dsNsp1 EVs; 8 and 9—20 bp marker.

FIG. 3. The cassette for expression of dsNsp1 RNA and Dcr1 protein fused with Sur7.

FIG. 4. Northern blot analysis of nsp1 dsRNA in Sb-Sur7-DCR1-dsNsp1 yeast cells and EVs, membrane hybridized with probes for nsp1 gene. Gel lines: 1—miRNA from Sb-Sur7-DCR1-dsNsp1 cells; 2—miRNA from Sb-Sur7-DCR1-dsNsp1 EVs; 3—20 bp marker.

FIG. 5. Schematic diagram of kinetics related to EV-mediated delivery of RNA from a yeast host cell to a target cell.

### DETAILED DESCRIPTION OF THE INVENTION

The present invention is directed to the direct expression of shRNA molecules in yeast, which are further processed into siRNA molecules in vivo, and packaged into extracellular vesicles (EVs) and delivered to a target organism causing the downregulation of a target gene. (Production and targeting of inhibitory RNA molecules utilizing yeast-derived EVs to downregulated gene expression in a target cell, and in particular target genes of SARS-CoV-2 coronavirus, are described in PCT/US2022/014958, which is incorporated herein by reference.)

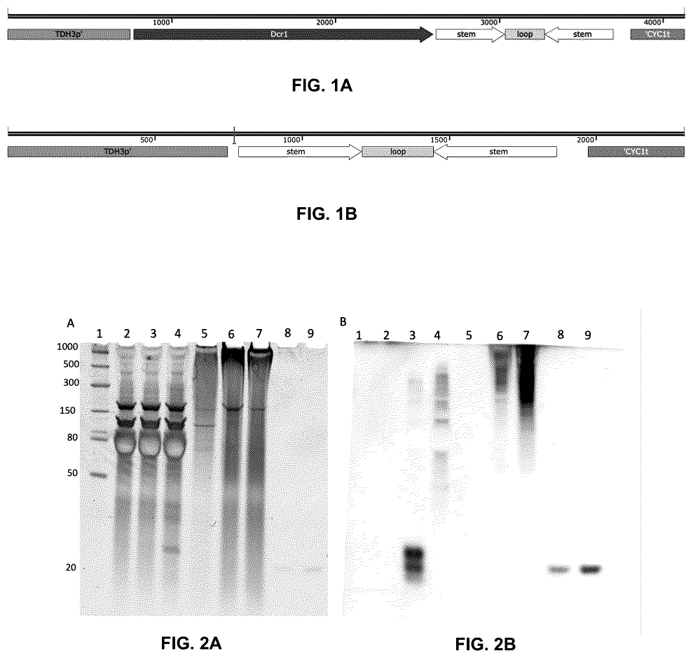

As shown in FIG. 1, the direct generation of siRNAs in yeast has several distinct advantage compared to the production and delivery of shRNAs to a target. Specifically, it avoids the viral-induced inhibition of the target host strain dicer enzymes thus increasing the efficacy of RNA interference in the host, which may preferably be a human subject (Maillard et al. 2019). In addition, it has been shown in several hosts that direct delivery of siRNAs is a more effective means of gene silencing than delivery of dsRNAs or shRNAs precursor RNAs that must be processed by the host machinery into siRNAs (Maillard et al. 2019). Finally, siRNAs are anticipated to have higher diffusion rates then shRNAs due to their smaller molecular weight and therefore enhance the kinetics of RNAi.

The present invention include systems, methods, and compositions to directly produce siRNAs in selected yeast strains, such as S. cerevisiae or S. boulardii. In one preferred embodiment, a yeast cell may be engineered to by an expression vector having a nucleotide sequence, operably linked to a promoter, encoding the S. castellii dicer gene (SEQ ID NO. 2. amino acid sequence according to SEQ ID NO. 1), or a fragment or variant thereof, along with a nucleotide sequence, operably linked to the same or separate promoter, encoding long (˜250 bp) shRNAs that would be processed into siRNAs by the heterologous dicer enzyme and packaged into yeast-derived EVs for delivery to target organism or cell.

In particular, the present invention relates to the expression, processing and delivery of RNA interference molecules (RNAi) to target cells through yeast-derived EVs. As used herein, the term RNA is used as it is in the art and is intended to mean at least one ribonucleic acid molecule. In one embodiment, the RNA is intended to elicit a gene silencing response, or RNA interference (RNAi) in the target cell. For example, the RNA expressed and processed in a yeast cell may be double stranded (dsRNA). This dsRNA may be process in the yeast cell into small interfering RNA (siRNA) as described herein and delivered to the target cell.

For example, dsRNA may be heterologously expressed in a yeast cell and processed into siRNA that can be delivered to a target cell. As is well-known, siRNA generally is a 21-23 nucleotide duplex with a 2-nucleotide overhang on the 3′ region of each strand, i.e., there is a region of single-strandedness of 2 nucleotides on each 3′ region of each strand. In one embodiment, the heterologous dsRNA that is expresses to the yeast cell is a dsRNA that is an RNA duplex at least 30 base pairs or more in length, and preferably approximately 250 or more base pairs nucleotides in length (Crook et al. 2014). As used herein the term RNA duplex means a double-stranded RNA molecule. The duplex may be made from two separate strands that are complementary to one another in specific regions, or the duplex may be formed by one single strand that is internally complementary to itself such that it can fold back on itself to form the RNA duplex. In select embodiments, the dsRNA that is delivered using the methods and compositions of the claimed invention is siRNAs that can be a duplex of 12, 13, 14, 15, 16, 17, 18, 19, 20, 21, 22, 23, 24, 25, 26, 27, 28, 29 or even 30 or more nucleotides. In additional select embodiments, the siRNAs delivered to the target cells may or may not have one or two 3′ nucleotide overhangs, and, if present, the one or two nucleotide overhangs may separately and independently be zero, one, two, three or four nucleotides in length.

As is well-known, the use of dsRNA to silence gene expression is not limited to specific messenger RNA (mRNA) sequences for each targeted gene. Rather, one strand of the dsRNA should be perfectly complementary or predominantly complementary to a region of the target mRNA that is targeted for silencing. There are now well-established “rules” and guidelines for design of, for example, siRNA molecules that can target mRNAs for cleavage and therefore gene silencing. For example, one of skill may employ the Ui-Tei rule (Ui-Tei, K., et al., Nucleic Acids Res., 32:936-948(2004), incorporated by reference), the Reynolds rule (Reynolds, A., et al., Nat. Biotechnol., 22:326-330 (2004), incorporated by reference) or the Amarzguioui rule (Amarzguioui, M and Prydz, H., Biochem. Biophys. Res. Commun., 316:1050-1058 (2004), incorporated by reference) in designing siRNAs for deliver into the target cell.

Accordingly, the heterologous dsRNA expressed in a yeast cell may include siRNA having a sequence on one strand of the RNA duplex that follows the Ui-Tei rule, the Reynolds rule or the Amarzguioui rule. For example, an siRNA that has one strand following the Ui-Tei rule includes (a) an A or U at position 1, from 5′ terminus of siRNA guide strand, (b) a G or C at position 19, (3) having AU in four or more positions of 1-7 of the guide strand, and (d) no long GC stretches of ten or more nucleotides. Additional characteristics of the siRNA sequence may or may not include other aspects of designer siRNA sequences, such as but not limited to having a UU sequence for at least one of the 3′ overhangs, a GC content of between about 30% to about 60%, for example around 50% to 52%. Other characteristics of the siRNA sequence that is delivered to the target cells may or may not include those characteristics noted in Naito, Y. and Ui-Tei, K., Front. Genet., Vol 3, Article 102 (2012).

Generally speaking, the siRNA delivered to the target cell contains one strand, i.e., the guide strand, that is perfectly (100%) complementary to a small stretch, about 15-23 bases, to a sequence within the target mRNA. Thus, the siRNA delivered to the target cell is not necessarily limited to a specific nucleotide sequence, except that the siRNA that is delivered the cell after delivery may be designed to have one strand that is 100% complementary to between about 15-23 bases of a target mRNA.

In one embodiment, the RNA that is delivered to target cells is not chemically modified in vitro. In another embodiment, the RNA that is delivered to the target cells is crosslinked RNA, such as but not limited to crosslinked siRNA. Crosslinked siRNA derivatives are as described in U.S. Pat. No. 10,087,441, which is incorporated herein by reference in its entirety. Crosslinking can be employed to alter the pharmacokinetics of the composition, for example, to increase the half-life in the body. Thus, the invention includes delivery of siRNA derivatives that include siRNA having two complementary strands of nucleic acid, such that the two strands are crosslinked. For example, a 3′ OH terminus of one of the strands can be modified, or the two strands can be crosslinked and modified at the 3′ OH terminus. The siRNA derivative delivered to the target cells can contain a single crosslink, e.g., a psoralen crosslink. In some embodiments, the siRNA derivative has at its 3′ terminus a biotin molecule, e.g., a photocleavable biotin, a peptide, a peptidomimetic, a nanoparticle, organic compounds, e.g., a dye such as a fluorescent dye, or a dendrimer. Modifying siRNA derivatives in this way may improve EV uptake or enhance targeting activities of the resulting siRNA derivative as compared to the corresponding siRNA and may be useful for tracing the siRNA derivative in the cell or improving the stability of the siRNA derivative compared to the corresponding siRNA. As such, one skilled in the art can screen crosslinked siRNA derivatives that are modified with various methods to determine whether the crosslinked siRNA derivatives possesses improved properties while maintaining the ability to mediate RNAi as are generally known in the art.

In additional embodiments, the siRNA that is delivered to the target cells is an siRNA directed one or more target genes. Established siRNA databases provide specific sequences of siRNAs in humans and other mammals that are involved in regulation of gene expression. For example, the siRNAdb database located at (https://bio.tools/sirnadb), or the SiRNAMod database at (http://crdd.osdd.net/servers/sirnamod/), which are incorporated by reference) As used herein, the phrase “directed against” or “directed to” when used in conjunction with RNA means that the RNA comprises at least one strand that is designed to promote gene silencing for a target gene.

In some embodiments of the present invention, the invention may include a kit for the treatment of a disease or condition in a subject in need thereof. In this preferred embodiment, the kit of the invention may include a quantity of SGEVs containing one or more siRNAs directed to inhibit the expression of one or more pathogen genes, or one or more endogenous genes in a subject. The quantity of SGEVs may be provided in a container, or other suitable receptacle or a quantity of SGEVs pre-loaded into a device for administration to a subject, preferably in a standard or customizable dosage. Optionally, the kit of the invention may include instructions for use.

In another preferred embodiment, the kit of the invention may include one or more yeast cells or cultures expressing a heterologous nucleotide sequence, operably linked to a promoter, encoding an dsRNA oligonucleotide (generally referred to herein as an RNA, and preferably an dsRNA configured to be process by a co-expressed heterologous dicer according to SEQ ID NO. 1, 3-4, directed to inhibit the expression of one or more pathogen genes, or one or more endogenous genes in a subject. The quantity of more yeast cells or cultures may be provided in a container, or other suitable receptacle and may be used to seed a culture for growth in a fermenter. Optionally, the kit of the invention may include instructions for use and fermentation.

In specific embodiments, the present invention further includes system, methods, and compositions to engineer a yeast cell, such as S. boulardii to express the dicer, or dicer-like protein, which may preferably be selected from SEQ ID NO. 1, 3-4, or a fragment or variant thereof, or an amino acid sequence having at least 85% sequence identity to any of SEQ ID NO. 1, 3-4, and preferably using a strong gene promoter driving the expression of a hairpin construct containing a dsRNA, and preferably a long dsRNA of approximately 30-250 or more base pairs, configured to downregulate expression of a target gene. In one embodiment, the target gene may include a reporter gene, such as a green fluorescent protein (GFP) gene expressed in human cells. EVs may be isolated from the engineered S. boulardii strain to determine the quantity of siRNAs that are present in the EVs and what their sequence identity and abundance is to determine if there is any bias in loading specific siRNA sequences into EVs. The relative GFP silencing in human host cells using an equal number of EVs from the dicer plus shRNA expressing strain can be compared to the GFP silencing using EVs from a control strain lacking dicer expression but expressing anti-GFP shRNAs. Relative silencing can be assessed by quantification of GFP fluorescence intensity and by qPCR quantification of GFP mRNA levels over a series of exposure time intervals after application of various amounts of EVs to human cells expressing GFP. In this method, optimization of the heterologous expression of dicer (SEQ ID NO. 1, or 3, or 4), and shRNAs, as well as their enzymatic characteristics can be optimized for siRNA production and loading into EVs, and for their use in in vivo gene downregulation, preferably in a human subject.

The present invention further include systems, methods, and compositions to directly produce siRNAs in selected yeast strains, such as S. cerevisiae or S. boulardii. In one preferred embodiment, a yeast cell may be engineered to by an expression vector having a nucleotide sequence, operably linked to a promoter, encoding the S. castellii dicer gene (SEQ ID NO. 2), or a fragment or variant thereof, a gene encoding a dicer-like protein from S. uvarum (SEQ ID NO. 3), or a gene encoding dicer protein NDA1 from N. dairenensis (SEQ ID NO. 4), along with a nucleotide sequence, operably linked to the same or separate promoter, encoding a shRNAs, and preferably a long shRNA of approximately 30-250 or more base pairs, that would be processed into siRNAs by the heterologous dicer enzyme and packaged into yeast-derived EVs for delivery to target organism or cell. Notably, the direct generation of siRNAs in yeast avoids the viral-induced inhibition of the target host strain dicer enzymes thus increasing the efficacy of RNA interference in the host, which may preferably be a human subject. In addition, it has been shown in several hosts that direct delivery of siRNAs is a more effective means of gene silencing than delivery of dsRNAs or shRNAs precursor RNAs that must be processed by the host machinery into siRNAs. Finally, siRNAs are anticipated to have higher diffusion rates then shRNAs due to their smaller molecular weight and therefore enhance the kinetics of RNAi.

In additional embodiments, the present invention includes systems, methods, and compositions for the enhanced RNAi processing capabilities in selected yeast strains, such as S. cerevisiae or S. boulardii. In one preferred embodiment, a yeast cell may be engineered to by an expression vector having a nucleotide sequence, operably linked to a promoter, encoding the S. castellii dicer gene (SEQ ID NO. 2), or a fragment or variant thereof, a gene encoding a dicer-like protein from S. uvarum (SEQ ID NO. 3), or a gene encoding dicer protein NDA1 from N. dairenensis (SEQ ID NO. 4), or a fragment or variant of the same, along with a nucleotide sequence, operably linked to the same or separate promoter, encoding a shRNAs, and preferably a long shRNA of approximately 30-250 or more base pairs, that would be processed into siRNAs by the heterologous dicer enzyme and packaged into yeast-derived EVs for delivery to target organism or cell.

In some embodiments of the present invention, the processed and delivered RNA is an siRNA that is directed against at least one of the ORF1ab, ORF3a, ORF7a, ORF8, S protein, N protein, the RdRp protein or M protein ORF, or the 5′-nsp1 region or 5′UTR region of the Severe Acute Respiratory Syndrome-Related Coronavirus 2 (SARS-CoV-2) virus. The polynucleotide sequences of each of these ORFs or region of the SARS-CoV-2 virus are well-known. See National Center for Biotechnology and Information (NCBI) Accession Number NC_045512.2 (available on the world wide web at (ncbi.nlm.nih.gov/nuccore/NC_045512.2) and Wu, F., et al., Nature, 579 (7798):265-269 (2020) both of which are incorporated by reference.

In some embodiments of the present invention, the siRNA delivered to a target cell, may include a siRNA delivered to a target cell by SGEVs that was expressed as a dsRNA and processed into the siRNA in the yeast cell or in yeast cell EVs. In another embodiment, of the present invention, the siRNA delivered to a target cell may be directed to a target gene of SARS-CoV-2, may further include a shRNA according to SEQ ID NO. 10, that is process into an siRNA according to SEQ ID NO. 14, by a dicer according to SEQ ID NO. 1, 3-4, or a fragment or variant thereof, preferably embedded in an EV. In this preferred embodiment, the yeast cell may express a heterologous nucleotide sequence, operably linked to a promoter, having at least 98% sequence identity to the nucleotide sequence of SEQ ID NO. 13. In a preferred embodiment, the target cell may be infected with, or at risk of infection by SARS-CoV-2.

In some embodiments of the present invention, the dsRNA expressed in a yeast cell, may include a shRNA directed to inhibit expression of a target gene in a target cell by SGEVs derived from yeast cells, preferably a Sb yeast cells, transformed by, and expressing by a heterologous expression vector. In this preferred embodiment, the invention may include an expression vector configured to express an dsRNA oligonucleotide. The expression vector of the invention may include a heterologous nucleotide sequence, operably linked to a promoter, having a nucleotide sequence encoding a dsRNA according to SEQ ID NO. 13. In another embodiment. The expression vector of the invention may include a heterologous nucleotide sequence, operably linked to a promoter, having a nucleotide sequence encoding a dsRNA with at least 98% sequence identity to the nucleotide sequence of SEQ ID NO. 13. In a preferred embodiment, the target cell may be infected with, or at risk of infection by SARS-CoV-2. This dsRNA may be processed into siRNAs directed to the target gene in the yeast cell by a heterologous dicer enzyme, preferably a S. castellii dicer protein (SEQ ID NO. 1), or a fragment or variant thereof, a gene encoding a dicer-like protein from S. uvarum (SEQ ID NO. 3), or a gene encoding dicer protein NDA1 from N. dairenensis (SEQ ID NO. 4), or a fragment or variant of the same.

In some embodiments of the present invention, the RNA delivered to a target cell, may include into siRNAs directed to the target gene derived from a dsRNA directed to the target gene expressed in the yeast cell and processed by a heterologous dicer enzyme, preferably a S. castellii (SEQ ID NO. 1), or a fragment or variant thereof, a gene encoding a dicer-like protein from S. uvarum (SEQ ID NO. 3), or a gene encoding dicer protein NDA1 from N. dairenensis (SEQ ID NO. 4), or a fragment or variant of the same, that is fused to the C-terminus of a yeast EV membrane protein such as Sur7 (SEQ ID NO. 5) and/or a truncated Fet3 (SEQ ID NO. 9) or truncated Msb2 (SEQ ID NO. 8) protein, or the transmembrane domain of Glycoprotein A (SEQ ID NO. 7) such that the dicer enzyme is localized in the lumen of the EV to process shRNAs localized to EVs into siRNAs. These targeted siRNAs may be packaged and delivered to a target cell by SGEVs, and may preferably include siRNA according to SEQ NO. 9.

In one embodiment, one or more of the EVs of the invention may include a pharmaceutical compositions. A “pharmaceutical composition” of the invention include a composition of the invention and a pharmaceutically acceptable carrier. The term “pharmaceutically acceptable” as used herein, refer to molecular entities and compositions that do not produce adverse, allergic, or other untoward reactions when administered to an animal or a human. The term, “pharmaceutically acceptable carrier” as used herein, includes any and all solvents, or a dispersion medium including, but not limited to, water, ethanol, a polyol (for example, glycerol, propylene glycol, and liquid polyethylene glycol, stachyose, and the like), suitable mixtures thereof, and vegetable oils, coatings, isotonic and absorption delaying agents, liposomes, commercially available cleansers, and the like. Supplementary bioactive ingredients also can be incorporated into such carriers.

The terms “administer,” “administering,” or “administration” refers to injecting, implanting, absorbing, or ingesting one or more therapeutic fusion peptides of the invention, which may be part of a pharmaceutical composition.

A “therapeutically effective amount” of a compound, preferably a therapeutic fusion peptide, of the present invention or a pharmaceutical composition thereof is an amount sufficient to provide a therapeutic benefit in the treatment of a disease or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, and/or enhances the therapeutic efficacy of another therapeutic agent. A “therapeutically effective amount” may also mean “prophylactically effective amount” of a compound of the present invention, such as a fusion peptide that is configured to target tau aggregates, or components thereof, is an amount sufficient to prevent a disease or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A “pharmaceutical composition” or “pharmaceutical composition of the invention” refers to a composition of the invention, and preferably a therapeutic fusion peptide composition of the invention, or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients. In other embodiments, the pharmaceutical composition further comprises at least one additional anticancer therapeutic agent, such as through a co-treatment. As used herein, a “pharmaceutically acceptable carrier” refers to a carrier or diluent that does not cause significant irritation to an organism and does not abrogate the biological activity and properties of the administered composition of the invention. The pharmaceutical acceptable carrier may comprise any conventional pharmaceutical carrier or excipient. The choice of carrier and/or excipient will to a large extent depend on factors such as the particular mode of administration, the effect of the carrier or excipient on solubility and stability, and the nature of the dosage form. As also used herein, a “pharmaceutical composition” may include additional mechanisms to deliver a fusion peptide of the invention to a target cell, such as a neuronal cell in a subject. In one embodiment, viral vectors, such as adenovirus vectors and subviral particles for fusion peptide delivery may be included within the definition of pharmaceutical compositions generally. See Kron M W, Kreppel F. Adenovirus vectors and subviral particles for protein and peptide delivery. Curr Gene Ther. 2012 October; 12(5):362-73, for methods of peptide delivery by viral vectors, which is incorporated herein by reference) Suitable pharmaceutical carriers include inert diluents or fillers, water, and various organic solvents (such as hydrates and solvates). The pharmaceutical compositions may, if desired, contain additional ingredients such as flavorings, binders, excipients, and the like. Thus, for oral administration, tablets containing various excipients, such as citric acid may be employed together with various disintegrants such as starch, alginic acid and certain complex silicates and with binding agents such as sucrose, gelatin, and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulations, solution suspension, for parenteral injection as a sterile solution, suspension, or emulsion, for topical administration as an ointment or cream or for rectal administration as a suppository. The pharmaceutical composition may be in unit dosage forms suitable for single administration of precise dosages.

Also encompassed by the invention are kits (e.g., pharmaceutical packs). The kits provided may comprise a therapeutic fusion peptide composition (e.g., pharmaceutical or diagnostic composition) and a container (e.g., a vial, ampule, bottle, syringe, and/or dispenser package, or other suitable container). The kits provided may comprise antibodies that selectively bind a therapeutic fusion peptide (e.g., pharmaceutical or diagnostic composition) and a container (e.g., a vial, ampule, bottle, syringe, and/or dispenser package, or other suitable container). In some embodiments, provided kits may optionally further include a second container comprising an excipient (e.g., pharmaceutically acceptable carrier) for dilution or suspension of an inventive pharmaceutical composition or compound. In some embodiments, the therapeutic fusion peptide composition provided in the first container and the second container are combined to form one unit dosage form.

The term “endogenous” gene or protein means that said gene or protein is expressed from a gene naturally found in the genome of a eukaryotic cell. The term “heterologous” gene or protein means that said gene or protein is not expressed from a gene naturally found in the genome of a eukaryotic cell. As used herein, the term “gene” or “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example, a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

As used herein, the phrase “expression,” “gene expression” or “protein expression,” such as the level of includes any information pertaining to the amount of gene transcript or protein present in a sample, in a cell, in a patient, secreted in a sample, and secreted from a cell as well as information about the rate at which genes or proteins are produced or are accumulating or being degraded (e.g., reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information.” Such information may be given in the form of amounts per cell, amounts relative to a control gene or protein, in unitless measures, etc.

The term “expression levels” refers to a quantity reflected in or derivable from the gene or protein expression data, whether the data is directed to gene transcript accumulation or protein accumulation or protein synthesis rates, etc. Polypeptides encoded by a target molecule genes that may be targeted for expression inhibition, for example through an RNAi mediated process herein may reflect a single polypeptide or complex or polypeptides. Accordingly, in another embodiment, the invention provides a polypeptide that is a fragment, precursor, successor or modified version of a protein target molecule described herein. In another embodiment, the invention includes a protein target molecule that comprises a foregoing fragment, precursor, successor or modified polypeptide.

A “fusion” or “chimera” protein is a polypeptide produced when two heterologous nucleotide sequences or fragments thereof coding for two (or more) different polypeptides not found fused together in nature are fused together in the correct translational reading frame. As used herein, a “functional” polypeptide or “fragment” is one that substantially retains at least one biological activity normally associated with that polypeptide (e.g., nucleosome formation). In particular embodiments, the “functional” polypeptide or “fragment” substantially retains all of the activities possessed by the unmodified peptide. By “substantially retains” biological activity, it is meant that the polypeptide retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide).

A “domain” refers to a unit of a protein or protein complex, comprising a polypeptide subsequence, a complete polypeptide sequence, or a plurality of polypeptide sequences where that unit has a defined function. The function is understood to be broadly defined and can be ligand binding, catalytic activity or can have a stabilizing effect on the structure of the protein.

An “expression vector” or “vector” refers to a nucleic acid construct, which when introduced into a host cell, results in transcription and/or translation of a RNA or polypeptide, respectively. More specifically, the term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; can be regulatory in nature, etc. There are various types of vectors including virus, plasmid, bacteriophages, cosmids, and bacteria. Again, more specifically, “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.”

In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s). A polynucleotide sequence is operably linked to an expression control sequence(s) (e.g., a promoter and, optionally, an enhancer) when the expression control sequence controls and regulates the transcription and/or translation of that polynucleotide sequence.

A “variant,” or “isoform,” or “protein variant” is a member of a set of similar proteins that perform the same or similar biological roles. For example, fragments and variants of the disclosed polynucleotides and amino acid sequences of the invention encoded thereby are also encompassed by the present invention. By “fragment” is intended a portion of the polynucleotide or a portion of the amino acid sequence. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. In certain preferred embodiments, a variant may include a polynucleotide having between 80-99% homology to the reference polynucleotide, while retaining the described. function.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “engineered” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

The term “peptide tag” or “peptide linker” as used herein generally refers to a peptide or oligopeptide. There is no standard definition regarding the size boundaries between what is meant by peptide or oligopeptide but typically a peptide may be viewed as comprising between 2-20 amino acids and oligopeptide between 21-39 amino acids. Accordingly, a polypeptide may be viewed as comprising at least 40 amino acids, preferably at least 50, 60, 70 or 80 amino acids. Thus, a peptide tag or linker as defined herein may be viewed as comprising at least 12 amino acids, e.g. 12-39 amino acids, such as e.g. 13-35, 14-34, 15-33, 16-31, 17-30 amino acids in length, e.g. it may comprise or consist of 12, 13, 14, 15, 16, 17, 18, 19, 20, 21, 22 or 23 amino acids.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “introducing,” “administered” or “administering”, as used herein, refers to any method of providing a composition of EVs to a patient such that the composition has its intended effect on the patient. In one embodiment, EVs may be introduced to a patient in vivo, while in other alternative embodiments, EVs may be introduced to subject cells in vitro which may then be administered to a patient in vivo.

The term “patient,” or “subject” as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies. As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “expression cassette” refers to a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a non-translated RNA, in the sense or antisense direction. The expression cassette comprising the nucleotide sequence of interest may be chimeric, meaning that at least one of its components is heterologous with respect to at least one of its other components. The peptides of the invention of the present invention may be chimeric.

The expression cassette may also be one which is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter which initiates transcription only when the host cell is exposed to some particular external stimulus.

As used herein, a promoter region or promoter element refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences may be cis acting or may be responsive to trans acting factors. Promoters, depending upon the nature of the regulation, may be constitutive or regulated.

As used herein, “operably linked” refers to a functional arrangement of elements. A first nucleic acid sequence is operably linked with a second nucleic acid sequence when the first nucleic acid sequence is placed in a functional relationship with the second nucleic acid sequence. For instance, a promoter is operably linked to a coding sequence if the promoter effects the transcription or expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence. The term “promoter” or “regulatory element” refers to a region or nucleic acid sequence located upstream or downstream from the start of transcription and which is involved in recognition and binding of RNA polymerase and/or other proteins to initiate transcription of RNA. Promoters useful in the present methods include, for example, constitutive, strong, weak, tissue-specific, cell-type specific, seed-specific, inducible, repressible, and developmentally regulated promoters.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

To accomplish delivery of RNA to target cells, the methods and compositions of the present invention comprise extracellular vesicles (EVs), and preferably EV generated from Saccharomyces, such as S. boulardii or S. cerevisiae. EVs generated from Saccharomyces sometimes are also interchangeably referred to as Saccharomyces-generated extracellular vesicles (SGEVs). The term extracellular vesicles are membranous vesicles released from cells. The extracellular vesicles of the methods and compositions of the invention are composed of lipid bilayers that can envelope and carry cargo in its interior. The lipid bilayer of the EVs may also include proteins embedded therein. In some embodiments, the SGEVs of the compositions and methods of the present invention can be exosomes or ectosomes. As is well-known, exosomes are generally formed upon the endocytosis of multivesicular endosomes (MVEs) to form intraluminal vesicles (ILVs) which are subsequently released into the extracellular environment as exosomes, whereas ectosomes are assembled and released from the plasma membrane. Often, the primary structural feature distinguishing ectosomes and ectosomes is diameter. In some embodiments, the diameter of the SGEVs are between about 30 nm to about 180 nm, between about 50 nm to about 200 nm, between about 75 nm to about 250 nm, between about 100 nm to about 300 nm, between about 125 nm to about 350 nm, between about 150 nm to about 400 nm, between about 175 nm to about 450 nm, between about 200 nm to about 500 nm, between about 250 nm to about 550 nm, between about 300 nm to about 600 nm, between about 350 nm to about between about 650 nm, between about 400 nm to about 700 nm, between about 450 nm to about 750 nm, between about 500 to about 800 nm, between about 550 nm to about 850 nm, between about 600 nm to about 900 nm, between about 650 nm to about 950 nm, between about 700 nm to about 1000 nm, between about 750 nm to about 1050 nm, between about 800 nm to about 1100 nm, between about 850 nm to about 1150 nm or between about 900 nm to about 1200 nm. Thus, exosomes may comprise components on their membrane surface, including but not limited to proteins, glycoproteins, proteoglycans, carbohydrates and lipids, which may be used to direct cargo into to exosome.

As understood by the disclosure herein, Saccharomyces is a single-celled organism, but the term “extracellular vesicle,” as it relates to the SGEVs, refers to vesicles that are secreted from Saccharomyces into the local environment, such as, but not limited to cell culture medium and organisms that may have ingested or consumed or been administered the Saccharomyces secreting the vesicles containing the heterologously-expressed RNA. In one embodiment, the SGEVs are secreted from Saccharomyces cerevisiae or Saccharomyces boullardii.

The Saccharomyces are engineered to express one or more heterologous RNAs, and preferably dsRNAs that may be employed in methods of silencing target genes. In select embodiments, the invention relates to methods of gene silencing comprising administering the SGEVs of the present invention, comprising heterologous RNA, to a cell or population of cells that express a target gene. The SGEVs can deliver their foreign RNA cargo, comprising a nucleotide sequence that targets a target gene for silencing, to the target cells, thereby silencing the target gene.

As used herein, a target gene is a gene whose expression is to be selectively inhibited or “silenced.” This silencing is achieved by promoting the degradation of the mRNA of the target gene that is induced by the binding between the delivered RNA, e.g., a shRNA, miRNA, siRNA, and the mRNA of the target gene. One portion or segment of these molecules is an anti-sense strand that is substantially complementary to a portion, e.g., about 16 to about 40 or more nucleotides of the mRNA of the target gene. Any gene previously identified by genetics or by sequencing may represent a target. Target genes may include, viral structural genes, such as but not limited to, capsid proteins, envelope proteins and membrane fusion proteins, viral non-structural genes such as but not limited to, virus replicon genes and virus immunomodulatory genes, viral regulatory and/or accessory genes. Other target genes include nuclear-encoded developmental genes and regulatory genes as well as metabolic or structural genes or genes encoding enzymes. In one embodiment of the present invention, the gene to which the delivered RNA is targeting for silencing is a viral gene that is necessary for virus replication.

As used herein, the gene silencing need not be a complete silencing. In one embodiment, the silencing is a “complete” silencing in that the gene expression is completely suppressed such that there is no detectable expression of the target gene. In other embodiments, the silencing is not a complete silencing and, instead, the silencing is partial. A partial gene silencing means a reduction in expression of the target gene such that expression may still be detectable. A reduction of gene expression can be assessed by determining gene expression levels before and after treatment or administration of the SGEVs. Gene expression levels can be measured using well-known methods, including but not limited to, measuring protein expression levels of the target gene and measuring mRNA levels of the target gene. Measuring protein expression levels can be accomplished directly, e.g., Western Blot, ELISA, etc. or indirectly, e.g., protein activity, metabolite levels, etc. In one embodiment, gene expression levels are measured with “RNA-seq,” which is a well-known methodology for RNA profiling. See Wang, Z., et al., Nat Rev Genet., 10(1): 57-63 (2009), which is incorporated by reference. The levels of gene expression of a target gene in a cell or group of cells can be measured prior to administration of the SGEVs by culturing the cells and measuring gene expression levels from the cells in culture. Then the SGEVs can be administered to the cells in culture and target gene expression levels can be reassessed to determine changes in gene expression levels.

The term “administering” as used herein means that the SGEVs are brought into contact or the same environment as the target cells. For example, if the SGEVs are administered to a subject by a routine route of administration, such as but not limited to, oral, intravenous, topical, intraperitoneal, intramuscular, subcutaneous, intranasal or intradermal route. If the SGEVs are administered to cells in culture, for example to assess differential gene expression levels, the SGEVs can be added to the culture medium.

The polynucleotides of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequence which encodes the peptides may be identical to the coding sequence shown in the sequence listing, or that of any of the deposited clones, or may be a different coding sequence which, as a result of the redundancy or degeneracy of the genetic code, encodes the same fusion proteins as shown in the sequence listing.

The term “nucleotide sequence encoding a peptide” encompasses a nucleotide sequence which includes only coding sequences for the polypeptide, e.g., heterologous protein, as well as a polynucleotide which includes additional coding and/or non-coding sequences. Thus, for example, the polynucleotides of the present invention may encode for a peptide, e.g., a heterologous protein, or for a peptide having a pro-sequence or for a protein having both a pro-sequence and pre-sequence.

The polynucleotides of the present invention may also have the coding sequence fused in frame to, for example, a marker sequence which allows for identification of the polypeptide of the present invention. The marker sequence may be a GFP protein, a hexa-histidine tag to provide for purification of the fusion protein is used.

The invention also relates to vectors, including but not limited to, expression vectors comprising the polynucleotides encoding the fusion proteins of the present invention. Types of vectors for expression for proteins and fusion proteins are well known in the art. In one embodiment, the vector is an expression vector for protein expression in Saccharomyces. Yeast expression vectors are commercially available from manufacturers.

The present invention also relates to methods of making and using these Saccharomyces-generated EVs. In one embodiment, the methods of making the SGEVs of the present invention comprise introducing into the Saccharomyces the expression vector encoding a heterologous protein related to processing dsRNAs to siRNA, such as SEQ ID NO. 14, or a fragment or variant thereof, of the present invention to generate a host Saccharomyces cell. The host cell is then cultured under conditions to permit protein production from the vector encoding the fusion protein. In one embodiment, the host cells of the present invention Saccharomyces cerevisiae or Saccharomyces boullardii.

Culture conditions for culturing yeast host cells are well-known in the art. The continued culture of the host cell will permit production and secretion of the SGEVs into the cell culture environment, where they can be isolated from culture. Methods of isolating extracellular vesicles, such as exosomes, from cell culture media are well-known in the art and are reviewed in Li, P. et al., Theranostics, 7(3):789-804 (2017), which is incorporated by reference herein. Generally speaking, methods of isolating the SGEVs from culture include but are not limited to ultracentrifugation methods, ultrafiltration, size-based exclusion methods, immunoaffinity capture-based methods, precipitation methods, microfluidics-based methods or some combination thereof.

The heterologously expressed RNA, and preferably siRNAs, may be present in the SGEVs immediately isolated from culture. The foreign siRNA can also be introduced into the SGEVs by a number of different techniques. In select embodiments, the SGEVs are loaded with the foreign siRNA by electroporation or the use of a transfection reagent. Extrapolation of the voltages used for electroporation of cells to take into account the size of the exosomes would suggest that excessively high voltages would be required for electroporation of exosomes. Surprisingly however, it is possible to use electroporation to load exosomes with siRNA using voltages in the range of between about 20V/cm to 1000V/cm, for example 20V/cm to 100V/cm, with capacitance between about 25 μF and about 250 μF, for example between 25 μF and 125 μF.

In an alternative aspect of the present invention, it is possible to load the SGEVs with the heterologously expressed RNA, and preferably siRNAs using transfection agents. Despite the small size of the exosomes, conventional transfection agents can be used for transfection of exosomes with genetic material. In some embodiments, transfection reagents for use in accordance with the present invention include cationic liposomes.

The route of administration of the SGEVs includes, but is not limited to, topical, transdermal, intranasal, rectal, oral, subcutaneous, intravenous, intraarterial, intramuscular, intraosseous, intraperitoneal, epidural and intrathecal as disclosed herein. In one example SGEV's may be derived or isolated from a GRAS and/or probiotic yeast cell, such as Saccharomyces cerevisiae, and preferably Saccharomyces boullardii. For example, Saccharomyces boullardii probiotics, releasing wild type exosomes, have been shown to diminish disease severity by reducing the expression of inflammatory cytokines and stimulating the expression of anti-inflammatory cytokines in multiple organs including the lungs and cardiovascular system. Saccharomyces boullardii cells also have low immunogenicity and positively modulate host immune response in the presence of additional antigens. Sb is well established for genetic manipulation which allows the present inventors to engineer the Sb strain for expression and loading of specific siRNAs in exosomes. Cultivation of Sb is fast, low-cost, and easy to scale up using established procedures. Finally, the lipids present in EVs are natural and thus not likely to be cytotoxic when used therapeutically unlike artificial lipids frequently used to package mRNA for vaccines.

In specific embodiments, the oral administration of the SGEVs include administering engineered yeast, producing the SGEVs, as a probiotic. As used herein, a probiotic is a microorganism, such as a bacteria or yeast, generally recognized as safe for human or animal consumption. The probiotics of the present invention may or may not have additional health benefits to the consumer. In specific embodiments of the present invention, the probiotics is a Saccharomyces cerevisiae or a Saccharomyces boullardii. For example, Saccharomyces boullardii probiotics, releasing wild type exosomes, have been shown to diminish disease severity by reducing the expression of inflammatory cytokines and stimulating the expression of anti-inflammatory cytokines in multiple organs including the lungs and cardiovascular system. Saccharomyces boullardii cells also have low immunogenicity and positively modulate host immune response in the presence of additional antigens. Sb is well established for genetic manipulation which allows the present inventors to engineer the Sb strain for expression and loading of specific siRNAs in exosomes. Finally, Cultivation of Sb is fast, low-cost, and easy to scale up using established procedures The probiotic used in the methods of administering will be engineered to produce the SGEVs of the present invention.

As used herein, the term “RNAi molecules” “interfering RNA molecules” or “interfering RNA” or RNA molecules configured to mediate RNA interference generally refers to an RNA which is capable of inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNAi molecules include dsRNAs such as siRNAs, miRNAs and shRNAs, sgRNA, CRISPR RNA (crRNs). In one embodiment, the RNA silencing agent is capable of inducing RNA interference. In another embodiment, the RNA silencing agent is capable of mediating translational repression. As used herein, an RNA molecule or even RNAi molecule may further encompass lincRNA molecules as well as lncRNA molecules.

In some embodiments of the invention, the nucleic acid agent is a double stranded RNA (dsRNA). As used herein the term “dsRNA” relates to two strands of anti-parallel polyribonucleic acids held together by base pairing and containing a loop region of ssRNA of variable length and sequence identity to allow foldback of the RNA to form complementary dsRNA regions. The two strands can be of identical length or of different lengths, provided there is enough sequence homology between the two strands that a double stranded structure is formed with at least 60%, 70% 80%, 90%, 95% or 100% complementary over the entire length. According to an embodiment of the invention, there are no overhangs for the dsRNA molecule. According to another embodiment of the invention, the dsRNA molecule comprises overhangs. According to other embodiments, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. It will be noted that the dsRNA can be defined in terms of the nucleic acid sequence of the DNA encoding the target gene transcript, and it is understood that a dsRNA sequence corresponding to the coding sequence of a gene comprises an RNA complement of the gene's coding sequence, or other sequence of the gene which is transcribed into RNA.

The inhibitory RNA sequence can be greater than 90% identical or even 100% identical, to the portion of the target gene transcript. Alternatively, the duplex region of the RNA may be defined functionally as a nucleotide sequence that is capable of hybridizing with a portion of the target gene transcript under stringent conditions (e.g., 400 mM NaCl, 40 mM PIPES pH 6.4, 1 mM EDTA, 60 degrees C. hybridization for 12-hours; followed by washing). The length of the double-stranded nucleotide sequences complementary to the target gene transcript may be at least about 18, 19, 21, 25, 50, 100, 200, 300, 400, 491, 500, 550, 600, 650, 700, 750, 800, 900, 1000 or more bases. In some embodiments of the invention, the length of the double-stranded nucleotide sequence is approximately from about 18 to about 530, or longer, nucleotides in length.

The present teachings relate to various lengths of dsRNA, whereby the shorter version i.e., x is shorter or equals 50 bp (e.g., 17-50), is referred to as siRNA or miRNA. Longer dsRNA molecules of 30-600 are referred to herein as dsRNA, which can be further processed for siRNA molecules. According to some embodiments, the nucleic acid sequence of the dsRNA is greater than 15 base pairs in length. According to yet other embodiments, the nucleic acid sequence of the dsRNA is 19-25 base pairs in length, 30-100 base pairs in length, 100-250 base pairs in length or 100-500 base pairs in length. According to still other embodiments, the dsRNA is 500-800 base pairs in length, 700-800 base pairs in length, 300-600 base pairs in length, 350-500 base pairs in length or 400-450 base pairs in length. In some embodiments, the dsRNA is 400 base pairs in length. In some embodiments, the dsRNA is 750 base pairs in length. In a preferred embodiment, a dsRNA of the invention may be at least 30 base pairs in length.

The term “siRNA” refers to small inhibitory RNA duplexes (generally between 17-30 base pairs, but also longer e.g., 31-50 bp) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs, preferably at least 250 base pairs or longer, in triggering RNAi is theorized to result from providing Dicer with a substrate (27mer) instead of a product (21mer) and that this improves the rate or efficiency of entry of the siRNA duplex into RISC. It has been found that position of the 3′-overhang influences potency of a siRNA and asymmetric duplexes having a 3′-overhang on the antisense strand are generally more potent than those with the 3′-overhang on the sense strand. This can be attributed to asymmetrical strand loading into RISC, as the opposite efficacy patterns are observed when targeting the antisense transcript.

In certain embodiments, dsRNA can come from 2 sources; one derived from gene transcripts generated from opposing gene promoters on opposite strands of the DNA and 2) from fold back hairpin structures produced from a single gene promoter but having internal complimentary. For example, strands of a double-stranded interfering RNA (e.g., a siRNA) may be connected to form a hairpin or stem-loop structure (e.g., a shRNA). Thus, as mentioned, the RNA silencing agent may also be a short hairpin RNA (shRNA). The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. It will be recognized by one of skill in the art that the resulting single chain oligonucleotide forms a stem-loop or hairpin structure comprising a double-stranded region capable of interacting with the RNAi machinery.

As used herein, the phrase “microRNA (also referred to herein interchangeably as “miRNA”) or a precursor thereof” refers to a microRNA (miRNA) molecule acting as a post-transcriptional regulator. Typically, the miRNA molecules are RNA molecules of about 20 to 22 nucleotides in length which can be loaded into a RISC complex and which direct the cleavage of another RNA molecule, wherein the other RNA molecule comprises a nucleotide sequence, essentially complementary to the nucleotide sequence of the miRNA molecule. Typically, a miRNA molecule is processed from a “pre-miRNA,” or as used herein, a precursor of a pre-miRNA molecule by proteins, such as DCL proteins, and loaded onto a RISC complex where it can guide the cleavage of the target RNA molecules. Pre-microRNA molecules are typically processed from pri-microRNA molecules (primary transcripts). The single stranded RNA segments flanking the pre-microRNA are important for processing of the pri-miRNA into the pre-miRNA. The cleavage site appears to be determined by the distance from the stem-ssRNA junction (Han et al. 2006, Cell 125, 887-901, 887-901).

As used herein, a “pre-miRNA” molecule is an RNA molecule of about 100 to about 200 nucleotides, preferably about 100 to about 130 nucleotides, which can adopt a secondary structure comprising an imperfect double stranded RNA stem and a single stranded RNA loop (also referred to as “hairpin”), and further comprising the nucleotide sequence of the miRNA (and its complement sequence) in the double stranded RNA stem. According to a specific embodiment, the miRNA and its complement are located about 10 to about 20 nucleotides from the free ends of the miRNA double stranded RNA stem. The length and sequence of the single stranded loop region are not critical and may vary considerably, e.g., between 30 and 50 nucleotides in length. The complementarity between the miRNA and its complement need not be perfect, and about 1 to 3 bulges of unpaired nucleotides can be tolerated. The secondary structure adopted by an RNA molecule can be predicted by computer algorithms conventional in the art such as mFOLD. The particular strand of the double stranded RNA stem from the pre-miRNA which is released by DCL activity and loaded onto the RISC complex is determined by the degree of complementarity at the 5′ end, whereby the strand, which at its 5′ end, is the least involved in hydrogen bonding between the nucleotides of the different strands of the cleaved dsRNA stem, is loaded onto the RISC complex and will determine the sequence specificity of the target RNA molecule degradation. However, if empirically the miRNA molecule from a particular synthetic pre-miRNA molecule is not functional (because the “wrong” strand is loaded on the RISC complex), it will be immediately evident that this problem can be solved by exchanging the position of the miRNA molecule and its complement on the respective strands of the dsRNA stem of the pre-miRNA molecule. As is known in the art, binding between A and U involving two hydrogen bonds, or G and U involving two hydrogen bonds is less strong that between G and C involving three hydrogen bonds.

Naturally occurring miRNA molecules may be comprised within their naturally occurring pre-miRNA molecules, but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds. Some pre-miRNA scaffolds may be preferred over others for their efficiency to be correctly processed into the designed microRNAs, particularly when expressed as a chimeric gene wherein other DNA regions, such as untranslated leader sequences or transcription termination and polyadenylation regions are incorporated in the primary transcript in addition to the pre-microRNA.

According to the present teachings, the dsRNA molecules may be naturally occurring or synthetic. The dsRNA can be a mixture of long and short dsRNA molecules such as, dsRNA, siRNA, siRNA+dsRNA, siRNA+miRNA, or a combination of same.

In a preferred embodiment, one or more nucleic acid agents are designed for specifically targeting a target gene of interest. It will be appreciated that the nucleic acid agent can be used to downregulate one or more target genes (e.g., as described in detail above). If a number of target genes are targeted, a heterogenic composition which comprises a plurality of nucleic acid agents for targeting a number of target genes is used. Alternatively, the plurality of nucleic acid agents is separately formulated. According to a specific embodiment, a number of distinct nucleic acid agent molecules for a single target are used, which may be used separately or simultaneously (i.e., co-formulation) applied.

For example, in order to silence the expression of an mRNA of interest, synthesis of the dsRNA suitable for use with some embodiments of the invention can be selected as follows. First, the mRNA sequence is scanned including the 3′ UTR and the 5′ UTR. Second, the mRNA sequence is compared to an appropriate genomic database using any sequence alignment software, such as the BLAST software available from the NCBI server (https://blast.ncbi.nlm.nih.gov/Blast.cgi). Putative regions in the mRNA sequence which exhibit significant homology to other coding sequences are filtered out. Qualifying target sequences are selected as templates for dsRNA synthesis. Preferred sequences are those that have as little homology to other genes in the genome to reduce an “off-target” effect. It will be appreciated that the RNA silencing agent of some embodiments of the invention need not be limited to those molecules containing only RNA, but further encompasses chemically-modified nucleotides and non-nucleotides.

The terms “comprises”, “comprising”, are intended to have the broad meaning ascribed to them in U.S. Patent Law and can mean “includes”, “including” and the like.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference. The terminology used herein is for describing particular embodiments and is not intended to be limiting. As used herein, the singular forms “a,” “and” and “the” include plural referents unless the content and context clearly dictate otherwise. Thus, for example, a reference to “a” or “the” marker may include a combination of two or more such markers. Unless defined otherwise, all scientific and technical terms are to be understood as having the same meaning as commonly used in the art to which they pertain. For the purposes of the present invention, the following terms are defined above.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Establishing Multiple siRNA Expression System in S. boulardii

As previously discussed, S. boulardii lacks an RNA induced silencing activity associated with the lack of the dicer and argonaut genes. In order to silence targeted viral genes in the host organism it is also desirable not to inactivate dsRNA-induced activation of interferon-mediated viral suppression. In humans, dsRNA induces expression of the LGP2 gene which represses dicer activity and thus the turnover of viral dsRNA to produce siRNA. Direct delivery of siRNA via yeast EVs, however, would avoid the need for dicer mediated processing of a dsRNA substrate used for siRNA production. Therefore, packaging siRNA targeting viral gene suppression in yeast EVs would be of great therapeutic benefit. To generate exemplary siRNAs targeting a SARS-CoV-2 nsp1 gene expressed in human lung cell cultures, Applicants designed a yeast gene cassette for co-expression of long dsRNA substrate simultaneously with dicer (DCR1) gene from S. castellii (SEQ ID NO. 2) under control of TDH3 promoter and CYC1 terminator (FIG. 1A). For hairpin dsRNA expression, Applicants a gene construct encoding a 5″ sense and 3′ anti-sense stem of 420 bp complementary to the 5′ prime untranslated region (UTR) of the SARS-CoV2 nsp1 gene separated by 240 bp long loop sequence was generated (FIG. 1A) (SEQ ID NO. 10). As control for dicer activity, second vector was used that only expressed the dsRNA cassette without DCR1 under the control of a TDH3 promoter and CYC1 terminator (FIG. 1B). All expression cassettes were genome integrated into the YPRCt3 locus on XVI chromosome of yeast yielding strains Sb-DCR1-dsNsp1 and Sb-dsNsp1 It was previously shown that integration at this locus doesn't affect the cell growth and gene expression in S. boulardii (Durmusoglu et al, 2020).

### Example 2: Evaluation of dsRNA Expression in Engineered Yeast

To evaluate expression of the dsRNA cassette, Applicants extracted miRNA from yeast cells and isolated EVs from both engineered strains and wild-type strains and performed northern blot analyses using probes specific to the nsp1/SARS-CoV2 target gene (FIG. 2). Applicants found that in yeast expressing dsRNA largely only long RNA molecules, about 300-500 nt in length, were detected. In contrast, in yeast expressing DCR1 and Sb-dsNsp1 almost all dsRNA was processed into small RNAs about 20 nt length consistent with dicer processing of dsRNA into siRNA. However, no short siRNA molecules were present in EVs. Contrary, EVs isolated from the Sb-dsNsp1 strain lacking dicer contained high levels of dsRNA of approximately 500-1000 nt in size, corresponding to full length or unprocessed hairpin dsRNA. EVs isolated from Sb-DCR1-dsNsp1 strain also contained long dsRNA molecules of similar size, although the amount of dsRNA was significantly lower compared to EVs from Sb-DsNsp1 strain. Based on these data, Applicants concluded that in S. boulardii long dsRNA molecules are naturally packaging in EVs, while short siRNA-like molecules are retained in the cytoplasm.

### Example 3: Targeting DCR1 Protein to EVs for Processing of Packaged Long dsRNA to siRNA Directly Inside EVs

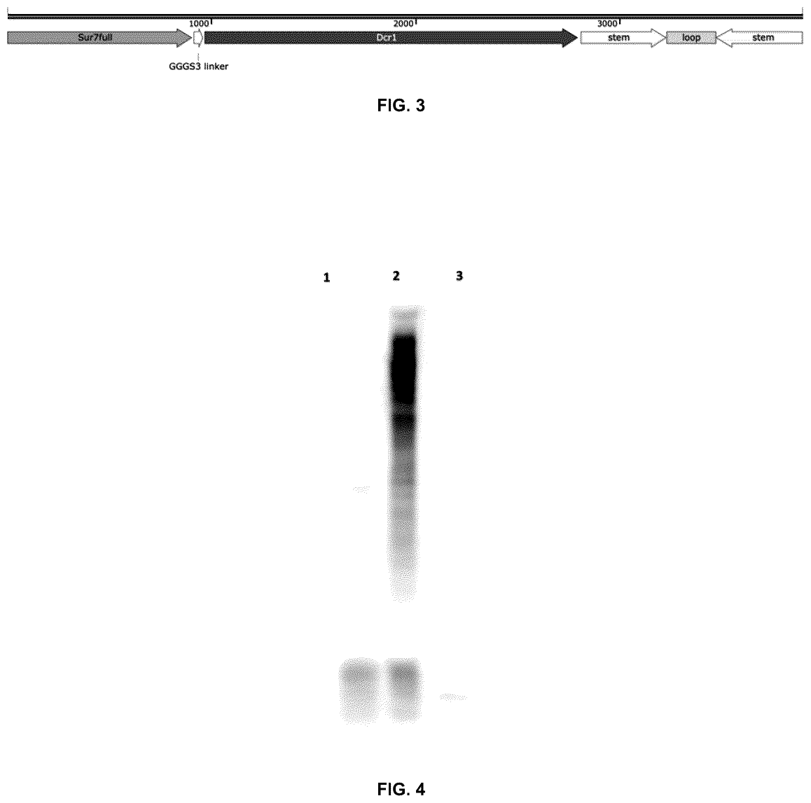

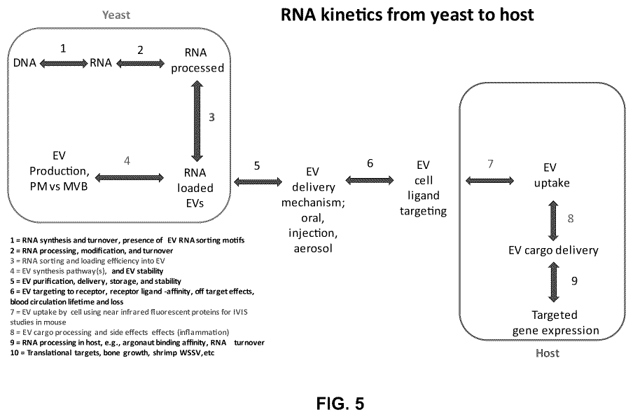

Since short dsRNA processed by dicer in the cytoplasm were not sorted into EVs, and only long dsRNA molecules were packaged into EVs, Applicants anchored dicer into EVs to process long dsRNA into siRNAs. Sur7 (SEQ ID NO. 5, nucleotide sequence according to SEQ ID NO. 6) is claudin-like transmembrane protein present in EVs isolated from C. albicans a close relative of S. boulardii (Dawson et al, 2020). Applicants fused the gene encoding to DCR1 to Sur7 via a C-terminal linker to process dsRNA sorted into EVs into siRNAs. According to prediction of topological structure and positioning of Sur7 protein, the C-terminal end of Sur7 is localized in the EV lumen, while N-terminal domain is extracellular. In order to target DCR1 inside EVs, Applicants expressed a Sur7-DCR1 fusion chimera, where DCR1 was connected to C-terminal end of Sur7 via flexible (GGGS)₃ linker (SEQ ID NO. 12) (FIG. 3).

To determine if the dicer gene fused to Sur7 would cleave dsRNA sorted into EVs, applicants performed comparative northern blot analysis of RNA extracted from Sb-Sur7-DCR1-dsNsp1 cells and isolated EVs (FIG. 4). Applicants found that the Sur7-dicer fusion protein processed dsRNA-nsp1 into siRNA that was compartmentalized in EVs. RNA extracted from EVs comprised both long dsRNA and short siRNA-like molecules 20 to 25 nt length. Thus, Applicants demonstrated that fusion of the dicer protein with an EV-localized anchor protein allowed cleavage of dsRNA in EVs into siRNA that was compartmentalized in the EVs.

### Example 4: Materials and Methods

S. boulardii strains design and construction: To create S. boulardii strains expressing DCR1 protein and dsRNA targeting the SARS-CoV-2 nsp1 gene, wild-type S. boulardii was transformed by dsDNA segments including DCR1-dsNsp1 expressing cassette and geneticin-resistance gene flanked on 5′ and 3′ ends by integration sequences homological to sequences from YPRCt3 locus on XVI chromosome. Transformation was performed by electroporation following the protocol described by Benatuil et al (2010)

EV isolation: Overnight cultures of Saccharomyces boulardii were diluted in 100 times with YPD medium. Cultures were then incubated for 24 h at 30° C. with shaking (200 rpm). For EVs isolation, cells and debris were removed by centrifugation at 3500×g for 35 min. EVs were concentrated from supernatant using tangential flow filtration device (Pall) with 300 kD membrane. Isolated EVs were aliquoted and stored at −80° C.

Northern blot analysis: RNA was extracted from yeast cells or isolated EVs using a mirVana miRNA isolation kit (Ambion). 6-15 pg of miRNA were loaded for separation on Novex™ 15% TBE-Urea gels along with ssRNA ladder (New England Biolabs). Afterward RNA was transferred onto a positively charged nylon transfer membrane (Whatman Nytran SuPerCharge, GE Healthcare Life Sciences, Germany) (Kim et al, 2010). Chemically synthesized RNA oligonucleotides were obtained from Integrated DNA Technologies, Inc. (USA, San Diego). RNA probes were labeled to high specific activity using a DIG Oligonucleotide 3′-End labeling kit, 2 Generation (Roshe Diagnostics GmbH, Germany). After UV cross-linking (UVP HL-2000 HybriLinker) the membranes were prehybridized at 42° C. for 30 min in an UL TRAhyb™-Oligo Hybridization Buffer (Thermo Fisher Scientific Baltics UAB, Lithuania). After prehybridization, the purified labeled probe was added to the prehybridization buffer and was incubated at 42° C. for 14-18 h. After hybridization, the membranes were washed twice with 2×SSC-0.2% SDS (20 min at 42° C.), 2×SSC-0.2% SDS (20 min at 55° C., twice), and with 1×SSC-0.1% SDS (20 min at 55° C., twice). The membranes were then washed and blocked with DIG Wash and Block Buffer Set, respectively (Roshe Diagnostics GmbH, Germany). DIG-labeled probes were detected using AP-coupled anti-digoxigenin Fab fragments (Roche Applied Science) diluted at a 1:100 ratio in alkaline phosphatase buffer and photoemission was detected using the ChemiDoc XRS+Imaging System (Bio-Rad). The signal intensities were quantified by densitometry using the Volume Tools of the Image Lab software, version 6.0.1 build 34 (Bio-Rad).

TABLE 1
Strains and cells lines

| Strains | Genotype | Origin |
|---|---|---|
| Saccharomyces boulardii | WT | ATCC type strain |
| | | MYA-796 |
| Sb-DCR1-dsNsp1 | DCR1-dsNsp1 | This invention |
| Sb-dsNSP1 | dsNsp1 | This invention |
| Sb-Su7- DCR1-dsNsp1 | Sur7- DCR1-dsNsp1 | This invention |

TABLE 2
Oligonucleotides

| Oligo | Sequence | Description |
|---|---|---|
| DH-p1 | ccacaagtgccatctttaagatgttgacgtgcctctgata (SEQ ID NO. 15) | Probe for detection in |
| | | northern blot analysis |
| DH-p2 | aacctctgaggcacctcctccagaatagtctccgtgcagt (SEQ ID NO. 16) | Probe for detection in |
| | | northern blot analysis |
| DH-p3 | cggagtctccaaagccacgtacgagcacgtcgcgaacctg (SEQ ID NO. 17) | Probe for detection in |
| | | northern blot analysis |
| DH-p4 | tgcaacacggacgaaaccgtaagcagcctgcagaagatag (SEQ ID NO. 18) | Probe for detection in |
| | | northern blot analysis |
| DH-p5 | ggacgaaacctagatgtgctgatgatcggctgcaacacgga (SEQ ID NO. 19) | Probe for detection in |
| | | northern blot analysis |
| DH-p6 | ccatcttacctttcggtcacacccggacgaaacctagatg (SEQ ID NO. 20) | Probe for detection in |
| | | northern blot analysis |
| Nsp1-m1 | cttaaagatggcacttgtgg (SEQ ID NO. 21) | Molecular weight marker |
| | | for northern blot |

### REFERENCES

1. Chatterjee, S., Fasler, M., Bussing, I., and Grosshans, H. (2011). Target-mediated protection of endogenous microRNAs in C. elegans. Dev. Cell 20, 388-396. doi: 10.1016/j.devcel.2011.02.008
2. Chen Y, Erpeng Guo, Jianzhi Zhang and Tong Si (2020) Advances in RNAi-Assisted Strain Engineering in Saccharomyces cerevisiae. Front. Bioeng. Biotechnol. doi: 10.3389/fbioe.200003
3. Crook, N. C., Schmitz, A. C., and Alper, H. S. (2014). Optimization of a yeast RNA interference system for controlling gene expression and enabling rapid metabolic engineering.

ACS Synth. Biol. 3, 307-313. doi: 10.1021/sb4001432
4. Drinnenberg, I. A., Weinberg, D. E., Xie, K. T., Mower, J. P., Wolfe, K. H., Fink, G. R. and Bartel, D. P. (2009) RNAi in budding yeast. Science, 326, 544-550.
5. Kildegaard, K. R., Tramontin, L. R. R., Chekina, K., Li, M., Goedecke, T. J., Kristensen, M., et al. (2019). CRISPR/Cas9-RNA interference system for combinatorial metabolic engineering of Saccharomyces cerevisiae. Yeast 36, 237-247. doi: 10.1002/yea.3390
6. Kim S W, Ii Z, Moore P S, Monaghan A P, Chang Y, Nichols M, John B. A sensitive non-radioactive northern blot method to detect small RNAs. Nucleic Acids Res. 2010 April; 38(7).e98. doi: 10.1093/nar/gkp1235. Epub 2010 Jan. 15. PMID: 20081203; PMCID: PMC2853138.
7. Orban, T. I., and Izaurralde, E. (2005). Decay of mRNAs targeted by RISC requires XRN1, the Ski complex, and the exosome. RNA 11, 459-469. doi: 10.1261/rna. 7231505
8. Lima, W. F., De Hoyos, C. L., Liang, X. H., and Crooke, S. T. (2016). RNA cleavage products generated by antisense oligonucleotides and siRNAs are processed by the RNA surveillance machinery. Nucleic Acids Res. 44, 3351-3363. doi: 10.1093/nar/gkw065
9. Maillard P V, van der Veen A G, Poirier E Z & Sousa C R (2019) Slicing and dicing viruses: antiviral RNA interference in mammals. The EMBO Journal 38: e100941
10. Purcell, O., Cao, J., Muller, I. E., Chen, Y. C., and Lu, T. K. (2018). Artificial repeat structured siRNA precursors as tunable regulators for Saccharomyces cerevisiae. ACS Synth. Biol. 7, 2403-2412. doi: 10.1021/acssynbio.8b00185
11. Seo G J, Kincaid R P, Phanaksri T, Burke J M, Pare J M, Cox J E, Hsiang T-Y, Krug R M, Sullivan C S (2013) Reciprocal inhibition between intracellular antiviral signaling and the RNAi machinery in mammalian cells. CellHost Microbe 14: 435-445.
12. Si, T., Luo, Y., Bao, Z., and Zhao, H. (2014). RNAi-assisted genome evolution in Saccharomyces cerevisiae for complex phenotype engineering. ACS Synth. Biol. 4, 283-291. doi: 10.1021/sb500074a
13. Suk, K., Choi, J., Suzuki, Y., Ozturk, S. B., Mellor, J. C., Wong, K. H., et al. (2011). Reconstitution of human RNA interference in budding yeast. Nucleic Acids Res. 39: e43. doi: 10.1093/nar/gkql321
14. Van der Veen A G, Maillard P V, Schmidt J M, Lee S A, Deddouche Grass S, Borg A, Kjær S, Snijders A P, Reis e Sousa C (2018) The RIG-I-like receptor LGP2 inhibits Dicer-dependent processing of long double-stranded RNA and blocks RNA interference in mammalian cells. EMBOJ37: e97479.
15. Volpe, T. A., Kidner, C., Hall, I. M., Teng, G., Grewal, S. I., and Martienssen, R. A. (2002). Regulation of heterochromatic silencing and histone H3 lysine-9 methylation by RNAi. Science 297, 1833-1837. doi: 10.1126/science.1074973
16. Williams, T. C., Averesch, N. J. H., Winter, G., Plan, M. R., Vickers, C. E., Nielsen, L. K., et al. (2015a). Quorum-sensing linked RNA interference for dynamic metabolic pathway control in Saccharomyces cerevisiae. Metab. Eng. 29, 124-134. doi: 10.1016/j.ymben.2015.03.008
17. Wilson, R. C., and Doudna, J. A. (2013). Molecular mechanisms of RNA interference. Annu. Rev. Biophys. 42, 217-239. doi: 10.1146/annurev-biophys-083012-130404
18. Weinberg, D. E., Nakanishi, K., Patel, D. J., and Bartel, D. P. (2011). The inside-out mechanism of Dicers from budding yeasts. Cell 146, 262-276. doi: 10.1016/j.cell.2011.06.021

What is claimed is:

1. An extracellular vesicle composition comprising:
a quantity of isolated yeast-generated extracellular vesicle (EVs) containing one or more heterologous small interfering RNAs (siRNAs); and
wherein said heterologous siRNAs were produced from a double-stranded RNA (dsRNA) heterologously expressed in the Saccharomyces cell, and further processed into said siRNAs by a heterologously expressed fusion peptide having a first dicer protein domain fused to an second EV membrane protein domain.

2. The composition of claim 1, wherein the yeast is selected from: Saccharomyces cerevisiae, or Saccharomyces boullardii.

3. The composition of claim 1, wherein the dsRNA comprises a long dsRNA.

4. The composition of claim 3, wherein long dsRNA is selected from:
a long dsRNA having between 30 and 250 more base pairs; or
a long dsRNA having more than 250 more base pairs.

5. The composition of any of claim 4, wherein the long dsRNA comprises a hairpin RNA (hpRNA).

6. The composition of any of claim 5, wherein said hpRNA comprises a hpRNA according to the nucleotide sequence SEQ ID NO. 10.

7. The composition of claim 1, wherein the first dicer protein domain comprises dicer protein from S. castellii, or a fragment or variant thereof.

8. The composition of claim 7, wherein the dicer protein comprises a protein according to the amino acid sequence SEQ ID NO. 1, or a fragment or variant thereof.

9. The pharmaceutical composition of claim 1, wherein said second EV membrane protein domain comprises a Sur7 protein from S. cerevisiae, or a fragment or variant thereof.

10. The composition of claim 9, wherein said Sur7 protein comprises a protein according to the amino acid sequence SEQ ID NO. 5, or a fragment or variant thereof.

11. The composition of claim 1, wherein said first dicer protein domain selected from:
a dicer protein from Saccharomyces castellii, or a fragment or variant thereof,
a dicer-like protein from Saccharomyces uvarum, or a fragment or variant thereof, and
a dicer protein from Naumovozyma dairenensis, or a fragment or variant thereof.

12. The composition of claim 1, wherein said first dicer protein domain selected from the amino acid sequences according to SEQ ID NO's. 1, 3, or 4, or a fragment or variant thereof, or an amino acid sequence having at least 85% sequence identify with an amino acid sequence according to SEQ ID NO's. 1, 3 or 4.

13. The composition of claim 1, wherein said second EV membrane protein domain comprises an EV membrane peptide selected from:
a Sur7 protein from S. boulardii, or a fragment or variant thereof
the transmembrane domain of Glycoprotein A, or a fragment or variant thereof;
a Msb2 protein from S. boulardii, or a fragment or variant thereof, and
a Fet3 protein from S. boulardii, or a fragment or variant thereof.

14. The composition of claim 1, wherein said second EV membrane protein domain comprises an EV membrane peptide selected from the amino acid sequences according to SEQ ID NO's. 5, 7-9, or a fragment or variant thereof or an amino acid sequence having at least 85% sequence identify with an amino acid sequence according to SEQ ID NO's. 5, 7-9.

15. The composition of claim 1, wherein said first dicer protein domain selected from: a dicer protein from S. castellii, or a fragment or variant thereof, a dicer-like protein from S. uvarum, or a fragment or variant thereof, and a dicer protein from N. dairenensis, or a fragment or variant thereof, and wherein said second EV membrane protein domain comprises an EV membrane peptide selected from a Sur7 protein from S. boulardii, or a fragment or variant thereof, the transmembrane domain of Glycoprotein A, a Msb2 protein from S. boulardii, or a fragment or variant thereof, and a Fet3 protein from S. boulardii, or a fragment or variant thereof.

16. The composition of claim 1, wherein said first dicer protein domain selected from the amino acid sequences according to SEQ ID NO's. 1, 3, or 4, or a fragment or variant thereof, or an amino acid sequence having at least 85% sequence identify with an amino acid sequence according to SEQ ID NO's. 1, 3 or 4, and wherein said second EV membrane protein domain comprises an EV membrane peptide selected from the amino acid sequences according to SEQ ID NO's. 5, 7-9, or a fragment or variant thereof or an amino acid sequence having at least 85% sequence identify with an amino acid sequence according to SEQ ID NO's. 5, 7-9.

17. The composition of any of claims 1, or 7-16, and further comprising a peptide linker linking with the first and said second domains of the fusion peptide.

18. The composition of claim 17, wherein said peptide linker comprises a (GGGS)₃ linker.

19. The composition of claim 1, wherein said siRNA comprises an siRNA according to the nucleotide sequence SEQ ID NO. 14.

20. A pharmaceutical composition comprising the EV of any of claims 1-19, and a pharmaceutically acceptable carrier.

21. A method delivering a siRNA to a target cell of a subject, the method comprising administering the pharmaceutical composition of claim 20 to the subject in need thereof.

22. A method delivering a siRNA to a target cell comprising contacting a target cell with a therapeutically effective amount of the EV of any of claims 1-19, and wherein said siRNA causes downregulation of the expression of one or more target genes.

23. The method of claim 21, wherein the steps of contacting comprises contacting a target cell with a therapeutically effective amount of the EV of any of claims 1-19, in vitro, ex vivo, or in vivo.

24. A kit containing the pharmaceutical composition of claim 20, a container, and instructions for use.

25. The kit of claim 24, wherein the a container contains a metered dose of the pharmaceutical composition.

26. A system for the production of extracellular vesicles containing heterologous RNA molecules comprising:
a yeast cell expressing heterologous nucleotide, operably linked to a promoter, encoding:
one or more heterologous double-stranded RNA (dsRNA);
fusion peptide having a first dicer protein domain fused to an second EV membrane protein domain; and
wherein fusion peptide is anchored in an extracellular vesicle (EVs) of the cell, and further processes the dsRNA into said small interfering RNAs (siRNAs).

27. The system of claim 26, wherein the isolated EVs are from Saccharomyces cerevisiae, or Saccharomyces boullardii.

28. The system of claim 26, wherein said promoter comprises a strong promoter.

29. The system of claim 28, wherein said strong promoter comprises TDH3.

30. The system of claim 26, wherein the dsRNA comprises a long dsRNA.

31. The system of claim 30, wherein long dsRNA is selected from:
a long dsRNA having between 30 and 250 more base pairs; or
a long dsRNA having more than 250 more base pairs.

32. The system of any of claim 31, wherein the long dsRNA comprises a hairpin RNA (hpRNA).

33. The system of any of claim 32, wherein said hpRNA comprises a hpRNA according to the nucleotide sequence SEQ ID NO. 10.

34. The system of claim 26, wherein the first dicer protein domain comprises dicer protein from S. castellii, or a fragment or variant thereof.

35. The system of claim 34, wherein the dicer protein comprises a protein according to the amino acid sequence SEQ ID NO. 1, or a fragment or variant thereof.

36. The system of claim 26, wherein said second EV membrane protein domain comprises a Sur7 protein from S. cerevisiae, or a fragment or variant thereof.

37. The system of claim 36, wherein said Sur7 protein comprises a protein according to the amino acid sequence SEQ ID NO. 6, or a fragment or variant thereof.

38. The system of claim 26, wherein said first dicer protein domain selected from:
a dicer protein from Saccharomyces castellii, or a fragment or variant thereof,
a dicer-like protein from Saccharomyces uvarum, or a fragment or variant thereof, and
a dicer protein from Naumovozyma dairenensis, or a fragment or variant thereof.

39. The system of claim 26, wherein said first dicer protein domain selected from the amino acid sequences according to SEQ ID NO's. 1, 3, or 4, or a fragment or variant thereof, or an amino acid sequence having at least 85% sequence identify with an amino acid sequence according to SEQ ID NO's. 1, 3 or 4.

40. The system of claim 26, wherein said second EV membrane protein domain comprises an EV membrane peptide selected from:
a Sur7 protein from S. boulardii, or a fragment or variant thereof
the transmembrane domain of Glycoprotein A, or a fragment or variant thereof;
a Msb2 protein from S. boulardii, or a fragment or variant thereof, and
a Fet3 protein from S. boulardii, or a fragment or variant thereof.

41. The system of claim 26, wherein said second EV membrane protein domain comprises an EV membrane peptide selected from the amino acid sequences according to SEQ ID NO's. 5, 7-9, or a fragment or variant thereof or an amino acid sequence having at least 85% sequence identify with an amino acid sequence according to SEQ ID NO's. 5, 7-9.

42. The system of claim 26, wherein said first dicer protein domain selected from: a dicer protein from S. castellii, or a fragment or variant thereof, a dicer-like protein from Saccharomyces uvarum, or a fragment or variant thereof, and a dicer protein from N. dairenensis, or a fragment or variant thereof, and wherein said second EV membrane protein domain comprises an EV membrane peptide selected from a Sur7 protein from S. boulardii, or a fragment or variant thereof, the transmembrane domain of Glycoprotein A, a Msb2 protein from S. boulardii, or a fragment or variant thereof, and a Fet3 protein from S. boulardii, or a fragment or variant thereof.

43. The system of claim 26, wherein said first dicer protein domain selected from the amino acid sequences according to SEQ ID NO's. 1, 3, or 4, or a fragment or variant thereof, or an amino acid sequence having at least 85% sequence identify with an amino acid sequence according to SEQ ID NO's. 1, 3 or 4, and wherein said second EV membrane protein domain comprises an EV membrane peptide selected from the amino acid sequences according to SEQ ID NO's. 5, 7-9, or a fragment or variant thereof or an amino acid sequence having at least 85% sequence identify with an amino acid sequence according to SEQ ID NO's. 5, 7-9.

44. The system of any of claims 26, or 34-43, and further comprising a peptide linker linking with the first and said second domains of the fusion peptide.

45. The system of claim 44, wherein said peptide linker comprises a (GGGS)₃ linker.

46. The system of claim 26, wherein said siRNA comprises an siRNA according to the nucleotide sequence SEQ ID NO. 14.

47. A method of producing extracellular vesicles containing siRNAs, the method comprising:
transforming a yeast host cell to express a heterologous nucleotide, operably linked to a promoter, encoding:
one or more heterologous double-stranded RNA (dsRNA);
a heterologous EV membrane protein linked to a dicer enzyme that processes said dsRNAs into siRNAs;
a dsRNA directed to inhibit the expression of more target genes in a host cell;
culturing the host cell under conditions that promote extracellular vesicle (EV) generation and
isolating the EVs from the culture, wherein said isolated EVs contain the one or more heterologous siRNAs.

48. The method of claim 47, wherein the isolated EVs are from Saccharomyces cerevisiae, or Saccharomyces boullardii.

49. The method of claim 47, wherein said promoter comprises a strong promoter.

50. The method of claim 49, wherein said strong promoter comprises TDH3.

51. The method of claim 47, wherein the dsRNA comprises a long dsRNA.

52. The method of claim 51, wherein long dsRNA is selected from:
a long dsRNA having between 30 and 250 more base pairs; or
a long dsRNA having more than 250 more base pairs.

53. The method of any of claim 52, wherein the long dsRNA comprises a hairpin RNA (hpRNA).

54. The method of any of claim 53, wherein said hpRNA comprises a hpRNA according to the nucleotide sequence SEQ ID NO. 10.

55. The method of claim 47, wherein the first dicer protein domain comprises dicer protein from S. castellii, or a fragment or variant thereof.

56. The method of claim 55, wherein the dicer protein comprises a protein according to the amino acid sequence SEQ ID NO. 1, or a fragment or variant thereof.

57. The method of claim 47, wherein said second EV membrane protein domain comprises a Sur7 protein from S. cerevisiae, or a fragment or variant thereof.

58. The method of claim 57, wherein said Sur7 protein comprises a protein according to the amino acid sequence SEQ ID NO. 6, or a fragment or variant thereof.

59. The method of claim 47, wherein said first dicer protein domain selected from:
a dicer protein from Saccharomyces castellii, or a fragment or variant thereof,
a dicer-like protein from Saccharomyces uvarum, or a fragment or variant thereof, and
a dicer protein from Naumovozyma dairenensis, or a fragment or variant thereof.

60. The method of claim 47, wherein said first dicer protein domain selected from the amino acid sequences according to SEQ ID NO's. 1, 3, or 4, or a fragment or variant thereof, or an amino acid sequence having at least 85% sequence identify with an amino acid sequence according to SEQ ID NO's. 1, 3 or 4.

61. The method of claim 47, wherein said second EV membrane protein domain comprises an EV membrane peptide selected from:
a Sur7 protein from S. boulardii, or a fragment or variant thereof
the transmembrane domain of Glycoprotein A, or a fragment or variant thereof;
a Msb2 protein from S. boulardii, or a fragment or variant thereof, and
a Fet3 protein from S. boulardii, or a fragment or variant thereof.

62. The method of claim 47, wherein said second EV membrane protein domain comprises an EV membrane peptide selected from the amino acid sequences according to SEQ ID NO's. 5, 7-9, or a fragment or variant thereof or an amino acid sequence having at least 85% sequence identify with an amino acid sequence according to SEQ ID NO's. 5, 7-9.

63. The method of claim 47, wherein said first dicer protein domain selected from: a dicer protein from S. castellii, or a fragment or variant thereof, a dicer-like protein from Saccharomyces uvarum, or a fragment or variant thereof, and a dicer protein from N. dairenensis, or a fragment or variant thereof, and wherein said second EV membrane protein domain comprises an EV membrane peptide selected from a Sur7 protein from S. boulardii, or a fragment or variant thereof, the transmembrane domain of Glycoprotein A, a Msb2 protein from S. boulardii, or a fragment or variant thereof, and a Fet3 protein from S. boulardii, or a fragment or variant thereof.

64. The method of claim 47, wherein said first dicer protein domain selected from the amino acid sequences according to SEQ ID NO's. 1, 3, or 4, or a fragment or variant thereof, or an amino acid sequence having at least 85% sequence identify with an amino acid sequence according to SEQ ID NO's. 1, 3 or 4, and wherein said second EV membrane protein domain comprises an EV membrane peptide selected from the amino acid sequences according to SEQ ID NO's. 5, 7-9, or a fragment or variant thereof or an amino acid sequence having at least 85% sequence identify with an amino acid sequence according to SEQ ID NO's. 5, 7-9.

65. The method of any of claims 47, or 55-64, and further comprising a peptide linker linking with the first and said second domains of the fusion peptide.

66. The method of claim 65, wherein said peptide linker comprises a (GGGS)₃ linker.

67. The method of any of claims 47-56, wherein said siRNA comprises an siRNA according to the nucleotide sequence SEQ ID NO. 14.


SEQUENCE LISTING (21 sequences)

SEQ ID NO: 1 - protein, 610 aa, Saccharomyces castellii
MNREKSADLSKTSDAPYNELDAKNLRNFYKVQNACAQLRESIKVIYENGLSSDQLNTMAKHGNDLEKSIANSPAMSVASCLNQVRPTLDIKNIFDHYKFENNSSPVDPYVHYPVCSDQNLENLAFIHRSLPNMNVKLTELQKTVMSNERLEFLGDSWLGALVAYIIYKKYPYANEGALSKMKEAIVNNNNLEKICEKLGFKERLKENIPRSSMKIKDRLTKNYADCVEAYIGALVIDRFSTEFNDVALWLEELSEEHFIELGPMMVKEPLNKNAKGELGAFLQFNNIGAKISYKRLNDKSPFKVEVRLGNNLLGIGDGSNVREAEQRAAMEALAQPKLIQKYSLHDIELERGMIEEADNVPQLLKSAEIPNQTPQSHHSITEEIDEEEGLTFPHSNPFLKGTPPAVLPEIVTNQIPQASADHQEQNVPDTDKIVNDVMERMSKILSVMVSEAVSNALGRPAPKNAPIPIPVTTPVSPLATSPVSVSVPAPAPAVAVTPSVVNNPPQPKMVTQPVSTNLNKTPEVALLNSVYDKEASGRLYALLGKYKLYPEYNTEQLGMTDFYTVCFIKGVGVEIGKGHGRSKKISQHKSAEDALNGKALKEYLRHCNNL

SEQ ID NO: 2 - DNA, 1830 bp, Saccharomyces castellii
  source 1..1830: mol_type = other DNA
atgaatagagaaaaaagcgccgatctaagtaaaacatcagatgctccatacaatgaactcgatgccaaaaatttaagaaatttctataaggttcaaaatgcatgtgcgcaattgagggaatccataaaagtaatttacgaaaacgggttatcatccgatcaattgaatactatggcgaaacatgggaacgacctagaaaaatctattgcaaacagtcctgctatgtccgtagcaagttgcttgaaccaagttagacccaccctagacatcaagaatatctttgatcattataaatttgaaaataattcatcaccagttgatccttatgtccattatcccgtatgttctgatcaaaatttggaaaatttggctttcattcatagatctttgccgaatatgaatgtcaaattgactgaattgcaaaagaccgtaatgagtaatgaacgtttggaatttttaggtgatagttggcttggcgccttagtggcgtatattatttataagaaatatccttatgctaatgaaggtgccttatcaaagatgaaggaagctatcgttaacaacaacaatttggaaaagatctgtgaaaaacttggctttaaagaaagattaaaggaaaatattccacgttcttcaatgaaaatcaaagatagattaaccaagaattacgcagactgtgtggaagcctacattggcgcattggttattgatagattttcaacagaatttaatgatgttgctctttggcttgaagaactatctgaggaacatttcattgaactaggacctatgatggtcaaagagcctttaaacaaaaatgcaaagggtgaactaggtgcgttcttacaatttaataatattggagccaaaatatcatataaaagattgaatgataaatctccgtttaaggtagaagtaagattggggaataatttgttaggtattggagatggttcaaatgtgagagaagctgaacagagagccgctatggaagcacttgctcaaccgaaattgattcaaaaatattctctacatgatattgaactagaaagaggcatgattgaagaagcagataatgttccccaattactcaaaagtgcggaaattcccaatcaaacaccacaaagtcatcattctatcacagaagaaattgatgaagaagaaggtcttacattcccccattcgaatccatttttgaaaggtacaccaccagcagttttacctgaaattgtaactaatcaaattccacaggcttctgcggatcatcaagaacaaaatgtgcctgacactgataaaattgtgaatgatgttatggaacgtatgagtaagatactgtcggtgatggtttctgaggctgtttcaaatgcacttggtagaccagctccaaaaaatgcacctatccctatcccagtaacaaccccagtctcaccgctagcaacatccccagtctcagtctcagtcccagcgccagctccagccgtagctgtcaccccatcagttgtgaataatccaccacaacccaaaatggtcacacaaccggtaagcacaaacctaaataaaacgcctgaagttgcacttttaaattctgtgtatgataaagaagcctcagggagactttatgcattgttaggtaaatataaattgtatccagaatataataccgagcaactaggaatgacagatttttatacggtatgctttataaagggtgttggtgtagaaattggtaaaggccatggtagaagtaaaaagatatcccaacataaatctgcagaagatgctttgaatggaaaagcactgaaggaatacttgaggcattgcaacaatctg

SEQ ID NO: 3 - protein, 591 aa, Saccharomyces uvarum
MKQYALEGGLFAGRDPLQLRRFYKIHNACVQLREAIKVIYDNAPTNEEINDMVKHGSRLEQSIGVSPAMSVASYLKTDQRSLDIESVFQRYKFENVDSPVHQFVHYPVVTNIHLENLAFVHRSVPNMNVNLTEVQKTVMSNERLEFLGDSWLGAFVAYILYRKYPFSDEGALSRMKSAIVNNNNLERLSQKLGFKERLKENIPRSKMRIKDKPFKHYADCVEAYIGALVIDRFSAEFVDVVDWIEELSQDQFNTLGGIMEKKPLNKNAKGELGNYLQFNKLGAKIKYFCLNNQPPFTSEVRLGDIFLGRGEGQNIKEAEQRAAMEAIDDIDIIQKYSIHDMEDRDSVYPYVEETHKTTGYNIGHTEIRSCSTDSIHGPGFTRGPKPNKSCKGLVTGSLPCLDDIISIKKTQKEIKEVQIAANYSSKYEDVDKEELMNEIMKRMETLLHPTVDDVLANMSISSQDHQCSTSFSTDISEPASSISFSSATASKSSPKGCNLAGDLPPILEPDSTGVDKIASDKLYAYMGRFHLTPKYTFKKLTNGSFHCVCTAGEFATTLGEGDAKRRKIAQHTAAAQALRSKVLVDIAHAQR

SEQ ID NO: 4 - protein, 749 aa, Naumovozyme dairenensis
MSVEPVFVISEQYTEKHGEELRSFYKVQNACVQLKEAIKIIYANAKPNEELVELFTNGNELEQAIAKSPAMSIASYLYSVQPSFDVESIFEHYAFHEDSTPVDQFIKYPVVSDASLENLAFIHRSLPNMNVNLTESQRTMMSNERLEFLGDSWLGALVAYSLYKKYPFANEGALSKMKQAIVNNDNLEKISKKLGFPEKLRGNIPHTSLRKKDKKSKLYADCVEAYIGALVVDRYSAEFKEVSQWLEDLSEDIFEEMGDSLTRDPLNKNSKGELAEILQFNKLGKKVEYKTLNKKSPFRVEVRLGNIVLGVGVGDNIKEAEQRSAMFALENPKELEKYTASPIESTSKVNNDLEYSPAYDDDESTLALNPAVDSPSEASDAMSSTSALVESVVKEVGDSGNVAVLIDTLMNRMSNVVEQMVSSTVSNILEKKGDVSVLTPELKANSTMGEVEAKNIKRTRKETTNNTQNMERIAKMDSIKKKVPPISYDEPDPNIIIINSESEDASESSFDDRRQKRVQTRNDLPNEAFINVDSNGIYKSSTNLKALQDFNALAAAYDAKGKKGRKNKGGQQDGNLVGGFDPAKDSNNRPGERKGRLAFGSRINNNPGNLEGRLGSSKKANTNNRPGKSAGGRSVPGMSGSINKTLGLKAGLDPSKDSNNRGGEKVPSSNNSTRYDKEASEKLYALLGKSNMCPEYETTNEGDGFYTVCRIKGMGITIGEGSGRSKKISQHIAAGNALRNKEVKKLLSR

SEQ ID NO: 5 - protein, 302 aa, Saccharomyces cerevisiae
MVKVWNIVLRLVVLLFLAGNTLLLILMIISGATDHYPVNRFYWVQGNTTGIPNAGDETRWTFWGACLQDKDGSDTCTSNLAPAYPISPVDNFNTHINVPHQFISKRDAFYYLTRFSFCFFWIALAFVGVSFILYVLTWCSKMLSEMVLILMSFGFVFNTAAVVLQTAASAMAKNAFHDDHRSAQLGASMMGMAWASVFLCIVEFILLVFWSVRARLASTYSIDNSRYRTSSRWNPFHREKEQATDPILTATGPEDMQQSASIVGPSSNANPVTATAATENQPKGINFFTIRKSHERPDDVSV

SEQ ID NO: 6 - DNA, 906 bp, Saccharomyces cerevisiae
  source 1..906: mol_type = other DNA
atggttaaggtctggaatatagtactacgtctggtagtgttgctgtttttagcaggtaataccttgctgttaattttgatgattatatctggtgccacggaccattaccccgtgaacagattctattgggtgcagggcaatacaacaggcatacctaatgctggggatgaaacacgttggacgttttggggcgcctgtctacaggataaagacgggtcggatacctgcacaagcaatttggcacctgcataccccatttccccagtagataacttcaacacacacatcaatgtccctcaccagtttatttccaaaagagacgctttttactacctgactagattttcattttgtttcttttggattgcgcttgcctttgtaggtgtgtcgtttatcctttacgttttgacttggtgctcgaagatgctttcagagatggtgcttatcctcatgtcttttgggtttgtcttcaatacggcagccgttgtcttgcaaacggccgcctctgccatggcaaagaatgctttccatgacgatcatcgtagtgcccaattgggtgcctctatgatgggtatggcttgggcaagtgtctttttatgtatcgtggaatttatcctgctggtcttctggtctgttagggcaaggttggcctctacttactccatcgacaattcaagatacagaacctcctccagatggaatcccttccatagagagaaggagcaagcaactgacccaatattgactgccactggacctgaagacatgcaacaaagtgcaagcatagtggggccttcttccaatgctaatccggtcactgccactgctgctacggaaaaccaacctaaaggtattaacttcttcactataagaaaatcacacgagcgcccggacgatgtctctgtt

SEQ ID NO: 7 - protein, 22 aa, Homo sapiens
ITLIIFGVMAGVIGTILLISYG

SEQ ID NO: 8 - protein, 174 aa, Saccharomyces boulardii
MQFPFACLLSTLVISGSLARASPFDFIFGNGGTLEYSSKSNSNVSTSSKSKKKIIGLVIGVVVGGCLYILFMIFAFKYIIRRRIQSQEIIKNPEISSISSSEFGGEKNYNNEKRMSVQESITQSMRIQNWMDDSYYGHGLTNNDSTPTRHNTSSSIPKISRPIASQNSLGWNEV

SEQ ID NO: 9 - protein, 67 aa, Saccharomyces boulardii
MTNTLLSIAVLLFSMLSLAQAETHTFNWTTGGFTKKGIIAMTFSCFAGILGIITIAIYGMMDMEDAT

SEQ ID NO: 10 - RNA, 1130 nt, synthetic construct
  source 1..1130: mol_type = other RNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagctcgagtctagaggaagcggaccacaagtgccatctttaagatgttgacgtgcctctgataagacctcctccacggagtctccaaagccacgtacgagcacgtcgcgaacctgtaaaacaggcaaactgagttggacgtgtgttttctcgttgaaaccagggacaaggctctccatcttacctttcggtcacacccggacgaaacctagatgtgctgatgatcggctgcaacacggacgaaaccgtaagcagcctgcagaagatagacgagttactcgtgtcctgtcaacgacagtaattagttattaattatactgcgtgagtgcactaagcatgcagccgagtgacagccacacagattttaaagttcgtttagagaacagatctacaagagatcgaaagttggttggtttgttacctgggaaggtataaacctttaatggcattcagtacggtcgtatattggagatacgaccgtactgaatgcctt

SEQ ID NO: 11 - DNA, 48 bp, synthetic construct
  source 1..48: mol_type = other DNA
agtggtggtggtggttctggtggtggtggttctggtggtggtggttct

SEQ ID NO: 12 - protein, 16 aa, synthetic construct
SGGGGSGGGGSGGGGS

SEQ ID NO: 13 - DNA, 49 bp, synthetic construct
  source 1..49: mol_type = other DNA
ggcattcagtacggtcgtatattggagatacgaccgtactgaatgcctt

SEQ ID NO: 14 - RNA, 49 nt, synthetic construct
  source 1..49: mol_type = other RNA
ggcattcagtacggtcgtatattggagatacgaccgtactgaatgcctt

SEQ ID NO: 15 - DNA, 40 bp, synthetic construct
  source 1..40: mol_type = other DNA
ccacaagtgccatctttaagatgttgacgtgcctctgata

SEQ ID NO: 16 - DNA, 40 bp, synthetic construct
  source 1..40: mol_type = other DNA
aacctctgaggcacctcctccagaatagtctccgtgcagt

SEQ ID NO: 17 - DNA, 40 bp, synthetic construct
  source 1..40: mol_type = other DNA
cggagtctccaaagccacgtacgagcacgtcgcgaacctg

SEQ ID NO: 18 - DNA, 40 bp, synthetic construct
  source 1..40: mol_type = other DNA
tgcaacacggacgaaaccgtaagcagcctgcagaagatag

SEQ ID NO: 19 - DNA, 41 bp, synthetic construct
  source 1..41: mol_type = other DNA
ggacgaaacctagatgtgctgatgatcggctgcaacacgga

SEQ ID NO: 20 - DNA, 40 bp, synthetic construct
  source 1..40: mol_type = other DNA
ccatcttacctttcggtcacacccggacgaaacctagatg

SEQ ID NO: 21 - DNA, 20 bp, synthetic construct
  source 1..20: mol_type = other DNA
cttaaagatggcacttgtgg